(19) United States
(12) Patent Application Publication
(10) Pub. No.: US 2022/0265657 A1
(43) Pub. Date: Aug. 25, 2022

(54) ADMINISTRATION METHOD AND DOSAGE REGIMEN FOR TREATMENT OF NEURODEGENERATIVE DISEASES USING TRAMETINIB AND MARKERS

(71) Applicant: GENUV INC., Seoul (KR)

(72) Inventors: Sung Ho HAN, Seoul (KR); Mi-Yeon KIM, Incheon (KR); Yoon Sun CHUN, Anyang (KR)

(21) Appl. No.: 17/612,899

(22) PCT Filed: May 22, 2020
(86) PCT No.: PCT/KR2020/006680

§ 371 Date: Nov. 19, 2021

Related U.S. Application Data
(60) Provisional application No. 62/851,488, filed on May 22, 2019.

(51) Int. Cl. A61K 31/519; C12Q 1/6883; G01N 33/573; G01N 33/68; A61P 25/28

(52) U.S. Cl. CPC A61K 31/519; C12Q 1/6883; G01N 33/573; C12Q 1/6851; A61P 25/28; C12Q 2600/106; C12Q 2600/158; G01N 33/6896

(57) ABSTRACT

The present invention relates to administration methods and dosage regimens for treatment of neurodegenerative diseases using trametinib and markers. The administration methods and dosage regimens induce neural regeneration and changes in gene expression.

### TECHNICAL FIELD

The present invention is directed to administration method and dosage regimen for the treatment of neurodegenerative diseases using trametinib. The present invention also includes a method for treating neurodegenerative diseases or other diseases associated with lysosomal dysfunction, autophagic flux, neuronal injury, damaged myelin or demyelination of nerve fibers, using trametinib and one or more makers whose level is changed by administration of trametinib.

### BACKGROUND ART

Neurodegenerative diseases such as Alzheimer's disease (AD) and Parkinson's disease (PD) are prevalent in the elderly population and the number of patients is increasing rapidly with the aging of society. Moreover, reports of early-onset types of neurodegenerative disease in the young are not uncommon. Thus, there is great interest in developing treatments that help stop the progress of the disease and in treatments that would restore damaged brain tissues.

The exact causes of such neurodegenerative diseases have not been established yet. According to what is known so far, neuronal cells in specific locations in the brain (e.g. the hippocampus or substantia nigra) are damaged, leading to a defective neural network among the reduced number of neuronal cells, which results in various symptoms of the neurodegenerative disease.

Research has been carried out in various fields to look for treatments. Although some drugs have been approved to relieve disease-associated symptoms in Alzheimer's disease, Parkinson's disease, and other neurodegenerative diseases, these drugs are limited to a short-term effect and have been associated with side effects. None repairs or restores damaged brain tissues. Recently, trametinib (SNR1611, MEKINIST®) was demonstrated to be effective in inducing neuronal differentiation and promoting survival of neurons and neural stem cells (NSCs), even in the presence of cytotoxic oligomers of Aβ₁₋₄₂ in vitro, as described in U.S. Pre-grant Pub. No. 2018/0169102, incorporated by reference in its entirety herein. Administration of trametinib and other MEK 1/2 inhibitors has therefore been suggested to be a method of protecting neurons against neuronal loss or damage and inducing neurogenesis, thus both treating the symptoms of and restoring brain tissues damaged by neurodegenerative diseases.

MEK 1/2 inhibitors were designed for use as anti-cancer agents, and the bulk of research on these agents has been in treatment of cancer. For therapeutic use of MEK 1/2 inhibitors for neurodegenerative diseases, especially for administration to elderly patients, there is therefore a need to develop appropriate administration methods and dosing regimens which will result in an effective treatment for neurodegenerative diseases with an acceptable side effect profile.

### DISCLOSURE OF INVENTION

### Technical Problem

The present disclosure relies on the discovery that administration of an effective amount of trametinib for more than four weeks can induce genetic, structural and functional changes associated with neural regeneration and enable the survival of differentiated neuron-like cells in the brain of Alzheimer Disease (AD) animal models. Since trametinib targets multiple pathways that promote the functional recovery of the degenerate cerebral neurons, these data predict that daily administration of an effective amount of trametinib for at least four weeks could reverse functional defects associated with neurodegenerative diseases and can be used for treatment of AD as well as other neurodegenerative diseases.

### Solution to Problem

Accordingly, in a first aspect, methods are presented for treating a neurodegenerative disease (e.g., AD) by administrating trametinib daily for at least four weeks.

In some embodiments, the method comprises the step of administering trametinib to a patient diagnosed with the neurodegenerative disease daily for at least four weeks.

In some embodiments, trametinib is administered for at least five weeks. In some embodiments, trametinib is administered for at least six weeks. In some embodiments, trametinib is administered for at least seven weeks. In some embodiments, trametinib is administered for at least eight weeks. In some embodiments, trametinib is administered for at least nine weeks. In some embodiments, trametinib is administered for at least three months.

In some embodiments, trametinib is administered at a daily oral dose effective to induce change in the level of one or more markers in the patient's brain or in a biological sample obtained from the patient of at least 1.3 fold after at least four weeks' administration as compared to prior to administration of trametinib. In some embodiments, the daily oral dose is effective to induce change in the level of the one or more markers in the patient's brain or in a biological sample obtained from the patient of at least 1.5 fold, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold.

In some embodiments, trametinib is administered at a daily oral dose effective to decrease the level of one or more markers in the patient's brain or in a biological sample obtained from the patient by at least 20% after at least four weeks' administration as compared to prior to administration of trametinib. In some embodiments, the daily oral dose is effective to decrease the level of the one or more markers in the patient's brain or in a biological sample obtained from the patient by at least 30%, at least 40%, at least 50%, at least 60%, at least 70%, at least 80%, at least 90%, or at least 99%.

In some embodiments, each of the one or more markers is encoded by a human homolog of the mouse gene selected from the group consisting of: Gabrb1, Gabrr2, Glra3, Nr3c2, Cdk15, Grin2a, Grin2b, Plcxd3, Chrm2, Chrna3, Chrna7, Chrnb2, Nefl, Pld1, Adra1a, Chrnb3, Slc6a3, Slc8a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Dcn, and Hmgb1. The human homologs of the mouse genes can be GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, DISC1, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6V0C, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

In some embodiments, the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcerebrosidase, β-glucuronidase. In some embodiments, the protease is a cathepsin. In some embodiments, the cathepsin is selected from the group consisting of: Cathepsin S, Cathepsin D, Cathepsin B, Cathepsin K, and Cathepsin L.

In some embodiments, trametinib is administered at a dose that provides a mean peak trametinib concentration (Cₘₐₓ) of at least 0.25 ng/g in the brain. In some embodiments, trametinib is administered at a dose that provides a mean peak brain trametinib concentration (Cₘₐₓ) of at least 0.5 ng/g in the brain. In some embodiments, trametinib is administered at a dose that provides a mean peak brain trametinib concentration (Cₘₐₓ) of at least 0.75 ng/g in the brain. In some embodiments, trametinib is administered at a dose that provides a mean peak brain trametinib concentration (Cₘₐₓ) of at least 1 ng/g in the brain. In some embodiments, trametinib is administered at a dose that provides a mean peak brain trametinib concentration (Cₘₐₓ) of at least 1.5 ng/g in the brain. In some embodiments, trametinib is administered at a dose that provides a mean peak brain trametinib concentration (Cₘₐₓ) of at least 2 ng/g in the brain. In some embodiments, trametinib is administered at a dose that provides a mean peak brain trametinib concentration (Cₘₐₓ) of at least 5 ng/g in the brain. In some embodiments, trametinib is administered at a dose that provides a mean peak brain trametinib concentration (Cₘₐₓ) of at least 10 ng/g in the brain. In some embodiments, trametinib is administered at a dose that provides a mean peak brain trametinib concentration (Cₘₐₓ) of at least 15 ng/g in the brain. In some embodiments, trametinib is administered at a dose that provides a mean peak brain trametinib concentration (Cₘₐₓ) of between 0.25 and 20 ng/g in the brain. In some embodiments, trametinib is administered at a dose that provides a mean peak brain trametinib concentration (Cₘₐₓ) of between 0.25 and 5 ng/g in the brain.

In some embodiments, trametinib is administered at an oral dose between 0.5 and 2 mg/day. In some embodiments, trametinib is administered at an oral dose greater than 0.5 and lower than 2 mg/day. In some embodiments, trametinib is administered at an oral dose greater than 0.75 and lower than 2 mg/day. In some embodiments, trametinib is administered at an oral dose greater than 1 and lower than 2 mg/day. In some embodiments, trametinib is administered at an oral dose greater than 0.75 and lower than 1.25 mg/day. In some embodiments, trametinib is administered at an oral dose of 0.5 mg/day. In some embodiments, trametinib is administered at an oral dose of 1 mg/day. In some embodiments, trametinib is administered at an oral dose of 1.5 mg/day. In some embodiments, trametinib is administered at a dose of 2 mg/day. In some embodiments, trametinib is administered as a tablet.

In some embodiments, the patient does not have BRAF V600E or V600K mutations. In some embodiments, the patient does not have cancer.

In some embodiments, the neurodegenerative disease is selected from the group consisting of Alzheimer's disease (AD), mild cognitive impairment (MCI), dementia, vascular dementia, senile dementia, frontotemporal dementia (FTD), Lewy body dementia (LBD), Parkinson's disease (PD), multiple system atrophy (MSA), corticobasal degeneration (CBD), progressive supranuclear palsy (PSP), Huntington's disease (HD), amyotrophic lateral sclerosis (ALS, Lou-Gehrig's disease), primary lateral sclerosis (PLS), progressive bulbar palsy (PBP), progressive muscular atrophy (PMA), pseudobulbar palsy, hereditary spastic paraplegia (HSP), cerebellar ataxia, Creutzfeldt-Jakob disease (CJD), multiple sclerosis (MS), and Guillain-Barre syndrome (GBS). In some embodiments, the neurodegenerative disease is Alzheimer's disease (AD).

In some embodiments, the method further comprises the step of detecting the level of one or more markers in a sample obtained from the patient. In some embodiments, each of the one or more markers is encoded by a human homolog of the mouse gene selected from the group consisting of: Gabrb1, Gabrr2, Glra3, Nr3c2, Cdk15, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Cxcl5, Rest, Sy2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Dcn, and Hmgb1.

In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcerebrosidase, β-glucuronidase. In some embodiments, the protease is a cathepsin. In some embodiments, the cathepsin is selected from the group consisting of: Cathepsin S, Cathepsin D, Cathepsin B, Cathepsin K, and Cathepsin L.

In some embodiments, the sample is obtained after the step of administering trametinib. In some embodiments, the sample is obtained at multiple time points after the step of administering trametinib. In some embodiments, the method further comprises the step of obtaining the sample. In some embodiments, the method further comprises the step of detecting the level of one or more markers in a control sample obtained from the patient before the step of administering trametinib. In some embodiments, the method further comprises the step of obtaining the control sample. In some embodiments, the sample is obtained by brain biopsy. In some embodiments, the sample is any biological sample obtained from an individual including body fluids, body tissue, cells, secretions, or other sources. In some embodiments, body fluids or secretions include blood, urine, saliva, stool, pleural fluid, lymphatic fluid, sputum, ascites, prostatic fluid, cerebrospinal fluid (CSF), or any other bodily secretion or derivative thereof. In some embodiments, blood is selected from whole blood, plasma, serum, peripheral blood mononuclear cells (PBMC), or any components of blood.

In some embodiments, the method further comprises the step of determining therapeutic efficacy of trametinib administered to the patient based on the change in the level of one or more markers. In some embodiments, the method further comprises the step of determining the duration or dose for subsequent administration of trametinib. In some embodiments, the method further comprises the step of discontinuing administration of trametinib based on determination of the therapeutic efficacy. In some embodiments, the method further comprises the step of continuing administration of trametinib based on determination of the therapeutic efficacy. In some embodiments, the method further comprises the step of adjusting administration of trametinib based on determination of the therapeutic efficacy. In some embodiments, the method further comprises: (a) detecting the level of the one or more markers in a biological sample obtained from the patient following administration of trametinib and (b) comparing the level detected in (a) with the level of the one or more markers in a biological sample obtained from the patient prior to administration of trametinib, or (c) comparing the level detected in (a) with the level of the one or more markers in a biological sample obtained from healthy subjects who are free of the disease(s) of interest.

In yet another aspect, the present invention discloses a method of enhancing lysosomal activity in a target tissue, comprising the step of administering trametinib to a subject, wherein the subject was diagnosed with a disorder associated with lysosomal dysfunction or autophagic flux.

In some embodiments, the disorder is selected from the group consisting of: lysosome storage disease, Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-I antitrypsin deficiency, dentatorubral pallidoluysian atrophy, frontal temporal dementia, progressive supranuclear palsy, x-linked spinobulbar muscular atrophy, neuronal intranuclear hyaline inclusion disease, multiple sclerosis, glaucoma and age-related macular degeneration.

In some embodiments, the lysosome storage disorder is selected from the group consisting of: alpha-mannosidosis, aspartylglucosaminuria, juvenile Neuronal Ceroid Lipofuscinosis (JNCL, juvenile Batten or CLN3 Disease), cystinosis, Fabry Disease, Gaucher Disease Types I, II, and III, Glycogen Storage Disease II (Pompe Disease), GM2-Gangliosidosis Type I (Tay Sachs Disease), GM2-Gangliosidosis Type II (Sandhoff Disease), Metachromatic Leukodystrophy, Mucolipidosis Types I, II/III and IV, Mucopolysaccharide Storage Diseases (Hurler Disease and variants, Hunter, Sanfilippo Types A, B, C, D, Morquio Types A and B, Maroteaux-Lamy and Sly diseases), Niemann-Pick Disease Types A/B, C1 and C2, Schindler Disease Types I and II.

In one aspect, the present disclosure provides a method of inducing axonogenesis in a target tissue, comprising the step of administering trametinib to a subject, wherein the subject was diagnosed with a disorder associated with neuronal injury.

In some embodiments, the disorder is selected from the group consisting of: glaucoma, stroke, head trauma, spinal injury, optic injury, ischemia, hypoxia, neurodegenerative disease, multiple sclerosis, and multiple system atrophy. In some embodiments, the disorder is selected from the group consisting of: diabetic neuropathies; virus-associated neuropathies; acquired immunodeficiency syndrome (AIDS) related neuropathy; infectious mononucleosis with polyneuritis; viral hepatitis with polyneuritis; Guillain-Barre syndrome; botulism-related neuropathy; toxic polyneuropathies including lead and alcohol-related neuropathies; nutritional neuropathies including subacute combined degeneration; angiopathic neuropathies including neuropathies associated with systemic lupus erythematosus; sarcoid-associated neuropathy; carcinomatous neuropathy; compression neuropathy (e.g. carpal tunnel syndrome); hereditary neuropathies, such as Charcot-Marie-Tooth disease; and peripheral nerve damage associated with spinal cord injury.

In some embodiments, the disorder is an ocular injury, ocular disorder, or optic neuropathy selected from the group consisting of: toxic amblyopia, optic atrophy, higher visual pathway lesions, disorders of ocular motility, third cranial nerve palsies, fourth cranial nerve palsies, sixth cranial nerve palsies, internuclear ophthalmoplegia, gaze palsies, eye damage from free radicals, ischemic optic neuropathies, toxic optic neuropathies, ocular ischemic syndrome, optic nerve inflammation, infection of the optic nerve, optic neuritis, optic neuropathy, papilledema, papillitis, retrobulbar neuritis, commotio retinae, glaucoma, macular degeneration, retinitis pigmentosa, retinal detachment, retinal tears or holes, diabetic retinopathy, iatrogenic retinopathy, and optic nerve drusen.

In one aspect, the present disclosure provides a method of treating a disease associated with damaged myelin or demyelination of nerve fibers, comprising the step of administering trametinib to a subject, wherein the subject was diagnosed with a disorder associated with damaged myelin or demyelination of nerve fibers.

In some embodiments, the disease is selected from the group consisting of: multiple sclerosis, acute disseminated encephalomyelitis, transverse myelitis, Schilder's disease, Balo's disease, clinically isolated syndrome, Alexander's disease, Canavan disease, Cockayne's syndrome, Pelizaeus-Merzbacher disease, optic neuritis, neuromyelitis optica, HTLV-I associated myelopathy, hereditary leukoencephalopathy, Guillain-Barre syndrome, central pontine myelinolysis, deep white matter ischemia, progressive multifocal leukoencephalopathy, demyelinating HIV encephalitis, demyelinating radiation injury, acquired toxic-metabolic disorders, posterior reversible encephalopathy syndrome, central pontine myelinolysis, leukodystrophies, adrenoleukodystrophy, Krabbe's globoid cell and/or metachromatic leukodystrophy. Other disease in which demyelination occurs include cervical spondylotic myelopathy resulting from cervical stenosis, traumatic injury to the brain or spinal cord, and hypoxic injury to the central nervous system including stroke and neonatal hypoxic injury.

In some embodiments of the aspects above, trametinib is administered for at least four weeks. In some embodiments, trametinib is administered for at least five weeks. In some embodiments, trametinib is administered for at least six weeks. In some embodiments, trametinib is administered for at least seven weeks. In some embodiments, trametinib is administered for at least eight weeks. In some embodiments, trametinib is administered for at least nine weeks. In some embodiments, trametinib is administered for at least three months.

In some embodiments, trametinib is administered at a daily oral dose effective to induce change in the level of one or more markers in the patient's target tissue or a biological sample obtained from the patient of at least 1.3 fold after the at least four weeks' administration as compared to prior to administration of trametinib. In some embodiments, the daily oral dose is effective to induce change in the level of the one or more markers in the patient's target tissue or a biological sample obtained from the patient of at least 1.5 fold, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold.

In some embodiments, trametinib is administered at a daily oral dose effective to decrease the level of one or more markers in the patient's target tissue or a biological sample obtained from the patient by at least 20% after administration of trametinib as compared to prior to the administration. In some embodiments, the daily oral dose is effective to decrease the level of one or more markers in the patient's target tissue or a biological sample obtained from the patient by at least 30%, at least 40%, at least 50%, at least 60%, at least 70%, at least 80%, at least 90%, or at least 99%.

In some embodiments, the one or more markers are encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Dcn, and Hmgb1.

In some embodiments, each of the one or more markers is selected from the group consisting of: β-hexosaminidase, β-galactosidase, β-galactosylcerebrosidase, β-glucuronidase. In some embodiments, the protease is a cathepsin. In some embodiments, the cathepsin is selected from the group consisting of: Cathepsin S, Cathepsin D, Cathepsin B, Cathepsin K, and Cathepsin L. Preferably, the protease is Cathepsin B.

In some embodiments, trametinib administration provides a mean peak trametinib concentration (Cₘₐₓ) of at least 0.25 ng/g in the target tissue. In some embodiments, the mean peak trametinib concentration (Cₘₐₓ) is at least 0.5 ng/g in the target tissue. In some embodiments, the mean peak trametinib concentration (Cₘₐₓ) is at least 0.75 ng/g in the target tissue. In some embodiments, the mean peak trametinib concentration (Cₘₐₓ) is at least 1 ng/g in the target tissue. In some embodiments, the mean peak trametinib concentration (Cₘₐₓ) is at least 1.5 ng/g in the target tissue. In some embodiments, the mean peak trametinib concentration (Cₘₐₓ) is at least 2 ng/g in the target tissue. In some embodiments, the mean peak trametinib concentration (Cₘₐₓ) is at least 5 ng/g in the target tissue. In some embodiments, the mean peak trametinib concentration (Cₘₐₓ) is at least 10 ng/g in the target tissue. In some embodiments, the mean peak trametinib concentration (Cₘₐₓ) is at least 15 ng/g in the target tissue. In some embodiments, the mean peak trametinib concentration (Cₘₐₓ) is between 0.25 and 20 ng/g in the target tissue. In some embodiments, the mean peak trametinib concentration (Cₘₐₓ) is between 0.25 and 5 ng/g in the target tissue.

In some embodiments, the target tissue is brain.

In some embodiments, trametinib is administered at an oral dose between 0.5 and 2 mg/day. In some embodiments, trametinib is administered at an oral dose greater than 0.5 and lower than 2 mg/day. In some embodiments, trametinib is administered at an oral dose greater than 0.75 and lower than 2 mg/day. In some embodiments, trametinib is administered at an oral dose greater than 1 and lower than 2 mg/day. In some embodiments, trametinib is administered at an oral dose greater than 0.75 and lower than 1.25 mg/day. In some embodiments, trametinib is administered at an oral dose of 0.5 mg/day. In some embodiments, trametinib is administered at an oral dose of 1 mg/day. In some embodiments, trametinib is administered at an oral dose of 1.5 mg/day. In some embodiments, trametinib is administered at a dose of 2 mg/day.

In another aspect, a pharmaceutical composition comprising trametinib is presented for use in the methods described above.

Another aspect of the invention provides a composition for use in determining the therapeutic efficacy of an MEK 1/2 inhibitor such as trametinib on a neurodegenerative disease, a disorder associated with lysosomal dysfunction or autophagic flux, a disorder associated with neuronal injury, or a disorder associated with damaged myelin or demyelination of nerve fibers, comprising a probe or an antibody that specifically binds to the one or more markers described above.

In some embodiments, the therapeutic efficacy of the MEK inhibitor is determined by comparing the level of the one or more markers in a biological sample obtained from a patient diagnosed with said disease or disorder after administration of trametinib (a) to the level of the one or more markers in a biological sample obtained from the patient prior to commencing administration of trametinib or (b) to the level of the one or more markers in a biological sample obtained from healthy subjects who are free of the disease or disorder.

Further aspect includes a method of detecting the level of the marker using a probe or an antibody that specifically binds to the one or more markers described above to provide information on the therapeutic efficacy of an MEK 1/2 inhibitor such as trametinib on a neurodegenerative disease, a disorder associated with lysosomal dysfunction or autophagic flux, a disorder associated with neuronal injury, or a disorder associated with damaged myelin or demyelination of nerve fibers.

### Advantageous Effects of Invention

The present disclosure relies on the discovery that administration of an effective amount of trametinib for more than four weeks can induce genetic, structural and functional changes associated with neural regeneration and enable the survival of differentiated neuron-like cells in the brain of Alzheimer Disease (AD) animal models. Since trametinib targets multiple pathways that promote the functional recovery of the degenerate cerebral neurons, these data predict that daily administration of an effective amount of trametinib for at least four weeks could reverse functional defects associated with neurodegenerative diseases and can be used for treatment of AD as well as other neurodegenerative diseases.

### BRIEF DESCRIPTION OF DRAWINGS

FIG. 1 shows brain (top) and plasma (bottom) concentration-time profiles of trametinib after single oral administration of trametinib in mice.

FIG. 2 provides a representative image of western blot analysis of pERKs and ERKs in mice whole brain lysates. ERKs were included as a loading control.

FIGS. 3A and B show data obtained from the brain lysate analysis of normal mice administered with trametinib in a time-dependent manner. FIGS. 3A and B trace upregulated genes (FIG. 3A) and down-regulated genes (FIG. 3B) related to biological processes in the brain at each time point in enriched gene ontology terms.

FIGS. 4A-C and 4D-G illustrate genes involved in synaptic activity, neurogenesis, lysosomal activity and autophagosome activity showing significant mRNA expression level changes by administration of trametinib compared to the vehicle treated group. The values in FIG. 4D-G represent fold change (FC) in the mRNA expression levels of the trametinib treated group compared to those of the vehicle treated group.

FIG. 5A shows normalized EPSC slope in LTP recordings from the CA1 recording electrode, measured at 3 min for baseline stabilization before TBS induction (red arrow) and following 20 min recording in WT-vehicle (black circle; n=8 slices from 6 mice), 5XFAD-vehicle (blue square; n=4 slices from 3 mice), and 5XFAD-trametinib (red triangle; n=6 slices from 3 mice). Representative EPSCs are displayed for each type with baseline (pale color) and response at 20 min (vivid color). Scalebar: 20 ms, 100 pA. FIG. 5B is a graph comparing average of normalized EPSC slopes from 15.5 min to 20 min.

FIG. 6A shows the average ratio of alternations in 3 minutes measured in the Y-maze test. FIG. 6B provides the average ratio of the number of investigations in 3 minutes measured and calculated in the novel object recognition test. P values were obtained by ANOVA test. *p<0.05, between WT-vehicle group (n=5), 5XFAD-vehicle group (n=4) and 5XFAD-trametinib group (n=5).

FIG. 7 provides immunofluorescence staining images and quantification of neurite/axon length and swollen axon area in the cortex layer V of 8-month old 5XFAD mice. 5-month old mice were administered with the vehicle and 0.1 mg/kg/day trametinib for 3 months. n=3 sagittal sections from each mouse, n=3 mice per group. Normalized to WT-vehicle group. Scale bars, 50 μm. Scale bars, 50 μm. P values were obtained by Student's t-test. *p<0.05, **p<0.005, and ***p<0.001 between WT-vehicle group and 5XFAD-vehicle group. #p<0.05. ##p<0.005, and, ###p<0.001 between 5XFAD-vehicle group and 5XFAD-trametinib group.

FIG. 8 provides immunofluorescence staining images and quantification of neurite/axon length and swollen axon area in the cortex layer V from 13-month old 5XFAD mice. Vehicle and trametinib were administered to 12-month old 5XFAD mice for 1 month. n=3 serial sagittal sections from each mouse, n=3 mice per group. Normalized to 5XFAD-vehicle group. Scale bars, 50 μm. P values were obtained by Student's t-test. *p<0.05, **p<0.005, and ***p<0.001 between 5XFAD-vehicle group and 5XFAD-trametinib group.

FIG. 9 is an image of representative western blot analysis of the brain cortex lysates from the mice of FIG. 7 for indicated proteins.

FIG. 10 is an image of representative western blot analysis of the brain cortex lysates from the mice of FIG. 8 for indicated proteins.

FIGS. 11A and B provide immunofluorescence staining images and quantification showing the change of dendritic spine by trametinib (SNR1611) in primary cortical neuron. P values were obtained by Student's t-test. *p<0.05, ***p<0.001 compared with the control group. ###p<0.001 compared with the Aβ₄₂-treated group (n=17).

FIG. 12 is an image of representative western blot analysis of mice brain cortex lysates for indicated proteins.

FIG. 13A is an image of representative western blot analysis of SH-SY5Y cell lysates for indicated proteins. FIG. 13B shows quantification of LC3II/LC3I (left) and mature cathepsin B (right) in the cells treated with trametinib (Tra) and/or Aβ₁₋₄₂ compared to non-treated control. P values were obtained by Student's t-test. *p<0.05, **p<0.005 compared with the control group. ###p<0.001 compared with the Aβ₄₂-treated group (LC3II/LC3I: n=5, cathepsin B: n=6).

FIG. 14A is an image of representative western blot analysis of the primary cortical neurons for indicated proteins. FIG. 14B shows quantification of mature cathepsin B in the neurons treated with trametinib (SNR1611) and/or Aβ₁₋₄₂ compared to non-treated control. P values were obtained by Student's t-test. *p<0.05 compared with the non-treated control group. #p<0.05 compared with the Aβ₄₂-treated group (n=5).

FIGS. 15A and B provide immunofluorescence images of LC3, LAMP1 and lysotracker and quantification of the co-stained ratio and number of lysotracker puncta of cells in SH-SY5Y cells. Scale bars, 10 μm. P values were obtained by Student's t-test. *p<0.05, **p<0.005 and ***p<0.001 between control vs. trametinib or control vs. Aβ₁₋₄₂. ##p<0.005 and ###p<0.001 between Aβ₁₋₄₂ vs. Aβ₁₋₄₂/trametinib.

FIGS. 16A and B are immunofluorescence images of LC3, LAMP1 and lysotracker (FIG. 16A), quantification of the cell ratio stained with both LC3 and LAMP1 antibodies (FIG. 16B top) and number of lysotracker puncta per cell (FIG. 16B bottom) in primary cortical neurons. Scale bars, 20 μm. P values were obtained by Student's t-test. *p<0.05, **p<0.005 and ***p<0.001 between control vs. trametinib or control vs. Aβ₁₋₄₂. #p<0.05, ##p<0.005 and ###p<0.001 between Aβ₁₋₄₂ vs. Aβ₁₋₄₂/trametinib.

FIGS. 17A, B and C are representative western blot analysis of SH-SY5Y cell lysates for indicated proteins.

FIG. 18A is an image of representative western blot analysis of primary cortical neurons for indicated proteins. FIGS. 18B and C show quantification of p-mTOR/mTOR and p-ULK1 (s757)/ULK1 in the neurons treated with trametinib (SNR1611) and/or Aβ₁₋₄₂ oligomer compared to non-treated control. P values were obtained by Student's t-test. *p<0.05 compared with the non-treated control. #p<0.05 compared with the Aβ₄₂-treated group (FIG. 18B; n=5, FIG. 18C; n=4).

FIG. 19 provides immunofluorescence images (left) and quantification (right) of apoptotic cells, LC3 and LAMP1 in cortex layer V. Arrows in the LC3/LAMP1 figures indicate co-stained regions. n=3 sagittal sections from each mouse, n=3 mice per group. Scale bars, 10 or 50 μm. P values were obtained by Student's t-test. #p<0.05 and ##p<0.005 between 5XFAD-vehicle group and 5XFAD-trametinib group, ***p<0.001 between WT-vehicle group and 5XFAD-vehicle group.

FIGS. 20A and B are the cathepsin B (CTSB) level in the plasma of 8-month old 5XFAD mice (FIG. 20A) and 13 month-old 5XFAD mice (FIG. 20B) after administration of trametinib (SNR0.05: trametinib 0.05 mg/kg/day, SNR0.1: trametinib 0.1 mg/kg/day) and donepezil. P values were obtained by Student's t-test. *p<0.05 compared with 5XFAD-vehicle group.

FIG. 21 is immunofluorescence staining images of Aβ, active caspase 3, and Tau in the NSCs from adult Tg2576 mice. The NSCs were treated with 100 nM of trametinib at undifferentiation or differentiation conditions for 48 hrs. Cell culture media conditions for undifferentiation contained 10 ng/ml EGF and 10 ng/ml bFGF. Growth factors were excluded in the condition for differentiation. Scale bars, 20 μm

FIGS. 22A and B provide immunofluorescence images of LAMP1, LC3 and the merge of the two signals in NSCs from adult Tg2576 mice (FIG. 22A) and magnification of the merged images (FIG. 22B). The NSCs were treated with 100 nM of trametinib at undifferentiation (“UD”) or differentiation (“D”) conditions for 48 hrs. Cell culture media conditions for undifferentiation contained 10 ng/ml EGF and 10 ng/ml bFGF. Growth factors were excluded in the condition for differentiation. Yellow arrows indicate the merged signals of LAMP1 and LC3. White arrowheads indicate LAMP1 signal only.

FIG. 23 shows myelin basic protein (MBP) staining in the cortex of 8-month old 5XFAD mice after administration of trametinib (SNR0.05: trametinib 0.05 mg/kg/day, SNR0.1: trametinib 0.1 mg/kg/day) and donepezil.

The figures depict various embodiments of the present invention for purposes of illustration only. One skilled in the art will readily recognize from the following discussion that alternative embodiments of the structures and methods illustrated herein may be employed without departing from the principles of the invention described herein.

### MODE FOR THE INVENTION

### 1. Definitions

Unless defined otherwise, all technical and scientific terms used herein have the meaning commonly understood by a person skilled in the art to which this invention belongs. As used herein, the following terms have the meanings ascribed to them below.

The term “MEK 1/2 inhibitor” as used herein refers to a compound that inhibits the function of both MEK 1 and MEK 2.

An exemplary MEK 1/2 inhibitor is trametinib (GSK-1120212, GSK1120212, JTP74057, or JTP-74057). The chemical name for trametinib is acetamide, N-[3-[3-cyclopropyl-5-[(2-fluoro-4-iodophenyl)aminol-3,4,6,7-tetrahydro-6,8-dimethyl-2,4,7-trioxopyrido[4,3-d]pyrimidin-1(2H)-yl]phenyl]. It has a molecular formula C₂₆H₂₃FIN₅O₄ with a molecular mass of 615.39. Trametinib has the chemical structure of Formula 1.

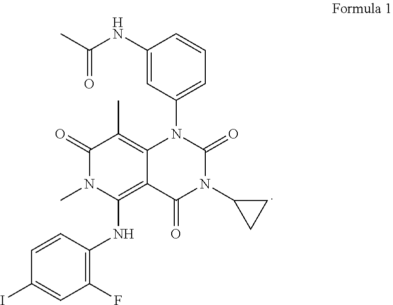 [SMILES: CC(=O)Nc1cccc(-n2c(=O)n(C3CC3)c(=O)c3c(Nc4ccc(I)cc4F)n(C)c(=O)c(C)c32)c1]

In the commercially available product, MEKINIST®, trametinib is in the form of a dimethyl sulfoxide solvate. In the inventions described herein, trametinib can be used in the form of a free base or a pharmaceutically acceptable salt or solvate, including the dimethyl sulfoxide solvate. Examples of possible solvates are hydrates, dimethyl sulfoxide, acetic acid, ethanol, nitromethane, chlorobenzene, 1-pentanol, isopropyl alcohol, ethylene glycol, 3-methyl-1-butanol, etc.

The term “therapeutically effective dose” or “effective amount” as used herein refers to a dose or amount that produces the desired effect for which it is administered. In the context of the present methods, a therapeutically effective amount is an amount effective to treat a symptom or improve a disease state of a subject with a neurodegenerative disease. The term “sufficient amount” as used herein refers to an amount sufficient to produce a desired effect.

### 2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 3. Methods of Treating a Neurodegenerative Disease

In a first aspect, methods are presented for treating patients with a neurodegenerative disease. The method comprises administering trametinib daily for at least four weeks to a patient diagnosed with neurodegenerative disease. In some embodiments, trametinib is administered to provide a mean peak trametinib concentration (Cₘₐₓ) of at least 0.25 ng/g in the brain.

Various delivery methods can be used to administer trametinib in the methods described herein. In currently preferred embodiments, trametinib is delivered by oral administration.

3.1. Subject for Treatment with Trametinib

3.1.1. Patients with a Neurodegenerative Disease

In the Examples below, we demonstrate that trametinib has multifaceted therapeutic actions that promote the functional recovery of degenerated cerebral neurons. Accordingly, the method described herein can be used for treatment of neurodegenerative diseases characterized by cortical degeneration, such as Alzheimer's disease. In the Examples below, we also show that trametinib facilitates lysosomal activity; accordingly, trametinib can be used in the treatment of diseases characterized by lysosomal dysfunction or autophagic flux dysfunction. In the Examples below, we show that trametinib induces axonogenesis (axogenesis) in the nervous system; accordingly, trametinib can be used in the treatment of a disease that can be controlled or cured by the induction of axonogenesis, such as diseases characterized by neuronal injury, including neuronal death, neurodegeneration, physically damaged nerve and/or neurite damage, axonopathy, and diminished potential for axonal growth. In the Examples below, we also show that trametinib protects or repairs myelin sheaths surrounding nerve cell axons; accordingly, trametinib can be used in the treatment of a disease associated with damaged myelin or demyelination of nerve fibers.

Neurodegenerative diseases that can be treated with the methods provided herein include, but are not limited to, dementia, vascular dementia, senile dementia, frontotemporal dementia (FTD), Lewy body dementia (LBD), Parkinson's disease (PD), multiple system atrophy (MSA), corticobasal degeneration (CBD), progressive supranuclear palsy (PSP), Huntington's disease (HD), amyotrophic lateral sclerosis (ALS, Lou-Gehrig's disease), primary lateral sclerosis (PLS), progressive bulbar palsy (PBP), progressive muscular atrophy (PMA), pseudobulbar palsy, hereditary spastic paraplegia (HSP), cerebellar ataxia, Creutzfeldt-Jakob disease (CJD), multiple sclerosis (MS), Guillain-Barre syndrome (GBS), and mild cognitive impairment (MCI).

In some embodiments, the patients selected for treatment have Alzheimer's disease (AD). The AD patient can have mild AD, moderate AD, or severe AD. In some embodiments, the patient has early-onset AD. In some embodiments, the patient has late-onset AD. In some embodiments, the AD patient exhibits high serum albumin to globulin ratio and high level of C-reactive protein, which are indicative of inflammation. In some embodiments, the AD patient does not exhibit elevated inflammatory biomarkers such as CRP or elevated serum albumin to globulin ratio. In some embodiments, the patient exhibits a deficiency of zinc throughout various regions of the brain. In some embodiments, the AD patient exhibits high plasma level of protease. In some embodiments, the protease is a cathepsin. In some embodiments, the cathepsin is selected from the group consisting of Cathepsin S, Cathepsin D, Cathepsin B, Cathepsin K, and Cathepsin L. In some embodiments, the protease is Cathepsin B.

In some embodiments, the patient has one or more symptoms, such as memory loss, language problems, unpredictable behavior, and personality and behavioral changes. In some embodiments, the patient does not have any behavioral symptom. In some embodiments, the patient has changes in one or more biomarkers associated with AD.

In some embodiments, the patient has mild cognitive impairment (MCI). In some embodiments, the patient has memory complaints and memory difficulties. In some embodiments, the patient has abnormal memory function documented by scoring below the education adjusted cutoff on the Logical Memory II subscale (Delayed Paragraph Recall) from the Wechsler Memory Scale—Revised (the maximum score is 25): a) less than or equal to 8 for 16 or more years of education, b) less than or equal to 4 for 8-15 years of education, c) less than or equal to 2 for 0-7 years of education. In some embodiments, the patient has Mini-Mental State Exam (MMSE) score between 24 and 30 (inclusive). In some embodiments, the patient's Clinical Dementia Rating is 0.5 and Memory Box score is at least 0.5. In some embodiments, the patient has general cognition and functional performance sufficiently preserved such that a diagnosis of AD cannot be made.

In some embodiments, the neurodegenerative disease involves abnormal activation of MAPK. In some embodiments, the neurodegenerative disease involves abnormal activation of the MAPK/ERK pathway. In some embodiments, the neurodegenerative disease involves abnormal endosomal-lysosomal function.

In preferred embodiments, the patient does not have the BRAF V600E or V600K mutation and the patient does not have cancer.

3.1.2. Patients with a Disorder Associated with Lysosomal Dysfunction or Autophagic Flux

In the Examples, we demonstrate that trametinib facilitates lysosomal activity by inducing autophagosome-lysosome fusion through the regulation of the mTOR (mammalian target of rapamycin) and TFEB (Transcription factor EB) pathways. Therefore, trametinib can be used in the treatment of diseases characterized by lysosomal dysfunction or autophagic flux dysfunction. Such diseases include, but are not limited to, lysosome storage disease, Alzheimer's disease, Parkinson's disease, amyotrophic lateral sclerosis. Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, neuronal intranuclear hyaline inclusion disease, multiple sclerosis, glaucoma and age-related macular degeneration. Lysosomal storage disease includes, but not limited to, alpha-mannosidosis, aspartylglucosaminuria, juvenile Neuronal Ceroid Lipofuscinosis (JNCL, juvenile Batten or CLN3 Disease), cystinosis, Fabry Disease, Gaucher Disease Types I, II, and III, Glycogen Storage Disease 11 (Pompe Disease), GM2-Gangliosidosis Type I (Tay Sachs Disease), GM2-Gangliosidosis Type II (Sandhoff Disease), Metachromatic Leukodystrophy, Mucolipidosis Types I, II/III and IV, Mucopolysaccharide Storage Diseases (Hurler Disease and variants, Hunter, Sanfilippo Types A, B, C, D, Morquio Types A and B, Maroteaux-Lamy and Sly diseases), Niemann-Pick Disease Types A/B, C1 and C2, Schindler Disease Types I and II.

3.1.3. Patients with a Disorder Associated with Neuronal Injury

In the Examples, we demonstrate that trametinib induces axonogenesis (axogenesis) in the nervous system. Thus, trametinib can be used in the treatment of a disease that can be controlled or cured by the induction of axonogenesis, such as diseases characterized by neuronal injury which includes but not is not limited to neuronal death, neurodegeneration, a physically damaged nerve and/or neurite damage, axonopathy, or diminished potential for axonal growth. Such diseases include, but are not limited to, glaucoma, stroke, head trauma, spinal injury, optic injury, ischemia, hypoxia, neurodegenerative disease, multiple sclerosis, and multiple system atrophy. Such diseases also include diabetic neuropathies; virus-associated neuropathies; including acquired immunodeficiency syndrome (AIDS) related neuropathy; infectious mononucleosis with polyneuritis; viral hepatitis with polyneuritis; Guillain-Barre syndrome; botulism-related neuropathy; toxic polyneuropathies including lead and alcohol-related neuropathies; nutritional neuropathies including subacute combined degeneration; angiopathic neuropathies including neuropathies associated with systemic lupus erythematosus; sarcoid-associated neuropathy; carcinomatous neuropathy; compression neuropathy (e.g. carpal tunnel syndrome); hereditary neuropathies, such as Charcot-Marie-Tooth disease; peripheral nerve damage associated with spinal cord injury. Such diseases also include an ocular injury or disorder (e.g. toxic amblyopia, optic atrophy, higher visual pathway lesions, disorders of ocular motility, third cranial nerve palsies, fourth cranial nerve palsies, sixth cranial nerve palsies, internuclear ophthalmoplegia, gaze palsies, eye damage from free radicals, etc.), or an optic neuropathy (e.g. ischemic optic neuropathies, toxic optic neuropathies, ocular ischemic syndrome, optic nerve inflammation, infection of the optic nerve, optic neuritis, optic neuropathy, papilledema, papillitis, retrobulbar neuritis, commotio retinae, glaucoma, macular degeneration, retinitis pigmentosa, retinal detachment, retinal tears or holes, diabetic retinopathy, iatrogenic retinopathy, optic nerve drusen, etc.).

3.1.4. Patients with a Disorder Associated with Damaged Myelin

In the Examples, we demonstrated that trametinib protects or repairs myelin sheaths surrounding nerve cell axons. Thus, trametinib can be used in the treatment of a disease characterized by damaged myelin or demyelination of nerve fibers, such as multiple sclerosis, acute disseminated encephalomyelitis, transverse myelitis, Schilder's disease, Balo's disease, clinically isolated syndrome, Alexander's disease, Canavan disease, Cockayne's syndrome, Pelizaeus-Merzbacher disease, optic neuritis, neuromyelitis optica, HTLV-I associated myelopathy, hereditary leukoencephalopathy, Guillain-Barre syndrome, central pontine myelinolysis, deep white matter ischemia, progressive multifocal leukoencephalopathy, demyelinating HIV encephalitis, demyelinating radiation injury, acquired toxic-metabolic disorders, posterior reversible encephalopathy syndrome, central pontine myelinolysis, leukodystrophies, adrenoleukodystrophy, Krabbe's globoid cell and/or metachromatic leukodystrophy. Other disease in which demyelination occurs include cervical spondylotic myelopathy resulting from cervical stenosis, traumatic injury to the brain or spinal cord, and hypoxic injury to the central nervous system including stroke and neonatal hypoxic injury.

3.2. Administration of Trametinib

3.2.1. Duration

The selected patient is administered a therapeutically effective amount of trametinib daily for at least four weeks. In some embodiments, trametinib is administered for a period sufficient to induce neural differentiation. In some embodiments, trametinib is administered for a period sufficient to induce neural regeneration. In some embodiments, trametinib is administered for a period sufficient to induce lysosomal activity. In some embodiments, trametinib is administered for a period sufficient to enhance autophagosome-lysosome fusion. In some embodiments, trametinib is administered for a period sufficient to induce axonogenesis. In some embodiments, trametinib is administered for a period sufficient to protect newly formed axons in the nervous system. In some embodiments, trametinib is administered for a period sufficient to induce repair or protection of myelin sheaths.

In certain embodiments, trametinib is administered for at least five weeks, for at least six weeks, for at least seven weeks, for at least eight weeks, for at least nine weeks, or for at least ten weeks. In certain embodiments, trametinib is administered for at least one month, for at least two months, for at least three months, or for at four months. In some embodiments, trametinib is administered for about six weeks, seven weeks, eight weeks, nine weeks, ten weeks or more. In some embodiments, trametinib is administered for about one month, two months, three months, four months, five months, six months, twelve months or more.

In some embodiments, trametinib is administered for a period sufficient to induce expression of genes involved in synaptic formation in the brain. In some embodiments, trametinib is administered for a period sufficient to induce expression of genes involved in neuroblast proliferation in the brain. In some embodiments, trametinib is administered for a period sufficient to induce expression of genes involved in axon growth in the brain. In some embodiments, trametinib is administered for a period sufficient to induce expression of genes involved in immune response in the brain. In some embodiments, trametinib is administered for a period sufficient to induce expression of genes involved in lysosomal and/or autophagosome activity. In some embodiments, trametinib is administered for a period sufficient to induce expression of genes involved in synaptic formation, neuroblast proliferation, axon growth, lysosomal activity and autophagosome activity in the brain.

In some embodiments, trametinib is administered until change in the level of one or more markers is detected. In some embodiments, each of the one or more markers is encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Dcn, and Hmgb1. In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcerebrosidase, β-glucuronidase. In some embodiments, the protease is a cathepsin. In some embodiments, the cathepsin is selected from the group consisting of: Cathepsin S, Cathepsin D, Cathepsin B, Cathepsin K, and Cathepsin L. The proteins can be used as a marker for measuring the efficacy of an MEK 1/2 inhibitor such as trametinib.

In some embodiments, trametinib is administered until the level of one or more markers reaches at least 1.3×, 1.5×, 2×, 3×, 4×, 5×, 6×, 7×, 8×, 9×, 10×, 20×, 30×, 40×, 50×, 100×, 200×, or 1000× of the levels measured prior to or without administration of trametinib. In some embodiments, trametinib is administered until the level of one or more markers reaches at most 0.8×, 0.7×, 0.6×. 0.5×, 0.4×, 0.3×, 0.2×, 0.1×. 0.05× or 0.01× of the levels measured prior to or without administration of trametinib.

In some embodiments, trametinib is administered until the level of one or more markers reaches at least 1.3×, 1.5×, 2×, 3×, 4×, 5×, 6×, 7×, 8×, 9×, 10×, 20×, 30×, 40×, 50×, 100×, 200×, or 1000× of a fixed or predetermined level. In some embodiments, trametinib is administered until the level of one or more s reaches at most 0.8×, 0.7×, 0.6×, 0.5×, 0.4×, 0.3×, 0.2×, 0.1×, 0.05×, or 0.01× of a fixed or predetermined level.

In some embodiments, trametinib is administered until a desired therapeutic outcome is detected. In some embodiments, the desired therapeutic outcome is change in behavioral symptoms of the patient. In some embodiments, trametinib is administered until unacceptable toxicity occurs.

3.2.2. Dose

Trametinib is administered at a therapeutically effective dose. In the methods described herein, the therapeutically effective dose is a dose effective to treat a neurodegenerative disease in the subject. In a particular embodiment, the therapeutically effective dose is a dose effective to treat AD in the subject.

In some embodiments, the therapeutically effective dose is the dose sufficient to induce neural differentiation. In some embodiments, the therapeutically effective dose is the dose sufficient to induce neural regeneration. In some embodiments, the therapeutically effective dose is the dose sufficient to induce lysosomal activity. In some embodiments, the therapeutically effective dose is the dose sufficient to induce axogenesis. In some embodiments, the therapeutically effective dose is the dose sufficient to enhance autophagosome-lysosome fusion in the subject. In some embodiments, the therapeutically effective dose is the dose sufficient to protect newly formed axons in the nervous system. In some embodiments, the therapeutically effective dose is the dose sufficient to induce repair or protection of myelin sheaths.

In some embodiments, the therapeutically effective dose is the dose sufficient to induce expression of genes involved in synaptic formation in the brain. In some embodiments, the therapeutically effective dose is the dose sufficient to induce expression of genes involved in neuroblast proliferation in the brain. In some embodiments, the therapeutically effective dose is the dose sufficient to induce expression of genes involved in axon growth in the brain. In some embodiments, the therapeutically effective dose is the dose sufficient to induce expression of genes involved in axogenesis. In some embodiments, the therapeutically effective dose is the dose sufficient to induce expression of genes involved in enhancing lysosomal activity. In some embodiments, the therapeutically effective dose is the dose sufficient to induce expression of genes involved in immune response in the brain. In some embodiments, the therapeutically effective dose is the dose sufficient to induce expression of genes involved in lysosomal and/or autophagosome activity. In some embodiments, the therapeutically effective dose is the dose sufficient to induce expression of genes involved in synaptic formation, neuroblast proliferation, axon growth, lysosomal activity and autophagosome activity in the brain.

In some embodiments, trametinib is administered in a dose sufficient to induce change in the level of one or more markers. In some embodiments, trametinib is administered in a dose sufficient to induce change in the level of one or more markers in the patient's target tissue or a biological sample obtained from the patient. In some embodiments, each of the one or more markers is encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Dcn, and Hmgb1. The human homologs of the mouse genes can be GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, DISC1, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6V0C, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1. In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcerebrosidase, β-glucuronidase. In some embodiments, the protease is a cathepsin. In some embodiments, the cathepsin is selected from the group consisting of: Cathepsin S, Cathepsin D, Cathepsin B, Cathepsin K, and Cathepsin L. The proteins can be used as a marker protein for measuring the effects of an MEK 1/2 inhibitor such as trametinib.

In some embodiments, trametinib is administered at a dose that provides a mean peak trametinib concentration (Cₘₐₓ) of at least 0.25 ng/g in the brain. In some embodiments, trametinib is administered at a dose that provides a mean peak trametinib concentration (Cₘₐₓ) of at least 0.5, 0.75, 1, 1.25, 1.50, 1.75, or 2 ng/g in the brain. In some embodiments, trametinib is administered at a dose that provides a mean peak trametinib concentration (Cₘₐₓ) of at least 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 ng/g in the brain. In some embodiments, trametinib is administered at a dose that provides a mean peak trametinib concentration (Cₘ) of about 4, 5, 6, 7, 8, 9, 10, 11, 12, 13, 14, 15, 16, 17, 18, 19 or 20 ng/g in the brain. In some embodiments, trametinib is administered at a dose that provides a mean peak trametinib concentration (Cₘₐₓ) of between 0.25 and 20, between 0.25 and 10, between 0.25 and 5, between 0.5 and 5, between 2.5 and 10, between 1 and 5 ng/g in the brain.

In some embodiments, trametinib is administered at a dose that provides a mean peak trametinib concentration (Cₘₐₓ) of at least 0.25 ng/ml in CSF. In some embodiments, trametinib is administered at a dose that provides a mean peak trametinib concentration (Cₘₐₓ) of at least 0.5, 0.75, 1, 1.25, 1.50, 1.75, or 2 ng/ml in CSF. In some embodiments, trametinib is administered at a dose that provides a mean peak trametinib concentration (Cₘₐₓ) of at least 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 ng/ml in CSF. In some embodiments, trametinib is administered at a dose that provides a mean peak trametinib concentration (Cₘₐₓ) of about 4, 5, 6, 7, 8, 9, 10, 11, 12, 13, 14, 15, 16, 17, 18, 19 or 20 ng/ml in CSF. In some embodiments, trametinib is administered at a dose that provides a mean peak trametinib concentration (Cₘₐₓ) of between 0.25 and 20, between 0.25 and 10, between 0.25 and 5, between 0.5 and 5, between 2.5 and 10, between 1 and 5 ng/ml in CSF.

In some embodiments, trametinib is administered at a dose that provides a mean peak trametinib concentration (Cₘₐₓ) of no more than 4.4 ng/g in the brain. In some embodiments, trametinib is administered at a dose that provides a mean peak trametinib concentration (Cₘₐₓ) of no more than 2 ng/g in the brain. In some embodiments, trametinib is administered at a dose that provides a mean peak trametinib concentration (Cₘₐₓ) of no more than 1.8 ng/g, no more than 1.6 ng/g, no more than 1.4 ng/g, no more than 1.2 ng/g, no more than 1 ng/g, no more than 0.8 ng/g, no more than 0.6 ng/g, or no more than 0.4 ng/g in the brain.

In some embodiments, trametinib is administered at a dose that provides a mean peak trametinib concentration (Cₘₐₓ) of no more than 4.4 ng/ml in CSF. In some embodiments, trametinib is administered at a dose that provides a mean peak trametinib concentration (Cₘₐₓ) of no more than 2 ng/ml in CSF. In some embodiments, trametinib is administered at a dose that provides a mean peak trametinib concentration (Cₘₐₓ) of no more than 1.8 ng/ml, no more than 1.6 ng/ml, no more than 1.4 ng/ml, no more than 1.2 ng/ml, no more than 1 ng/ml, no more than 0.8 ng/ml, no more than 0.6 ng/ml, or no more than 0.4 ng/ml in CSF.

In some embodiments, trametinib is administered at a dose that provides a mean peak trametinib concentration (Cₘₐₓ) of no more than 22.2 ng/ml in the plasma. In some embodiments, trametinib is administered at a dose that provides a mean peak trametinib concentration (Cₘₐₓ) of no more than 20 ng/ml, no more than 18 ng/ml, no more than 16 ng/ml, no more than 14 ng/ml, no more than 12 ng/ml, no more than 10 ng/ml, no more than 8 ng/ml, no more than 6 ng/ml, or no more than 4 ng/ml in the plasma.

In some embodiments, trametinib is administered at a dose that provides an area under the concentration curve (AUC) of trametinib in the brain of at least 25, 30, 35, 40, 45, 50, 55, 60, 65, 70, 75, 80, 85, 90, or 100 ng·h/g. In some embodiments, trametinib is administered at a dose that provides an area under the brain concentration curve of trametinib of about 25, 30, 35, 40, 45, 50, 55, 60, 65, 70, 75, 80, 85, 90, 95, 100, 110, 120, 130, 140, 150, 160, 170, 180, 190, 200, 250, or 300 ng·h/g. In some embodiments, trametinib is administered at a dose that provides an area under the brain concentration curve of trametinib of about 20 to about 700 ng·h/g, about 20 to about 600 ng·h/g, about 30 to about 500 ng·h/g, about 50 to about 400 ng·h/g, about 50 to about 300 ng·h/g, about 50 to about 200 ng·h/g, about 50 to about 100 ng·h/g, about 60 to 300 ng·h/g, about 30 to about 200 ng·h/g.

In some embodiments, trametinib is administered at a dose that provides an area under the concentration curve (AUC) of trametinib in CSF of at least 25, 30, 35, 40, 45, 50, 55, 60, 65, 70, 75, 80, 85, 90, or 100 ng·h/ml. In some embodiments, trametinib is administered at a dose that provides an area under the CSF concentration curve of trametinib of about 25, 30, 35, 40, 45, 50, 55, 60, 65, 70, 75, 80, 85, 90, 95, 100, 110, 120, 130, 140, 150, 160, 170, 180, 190, 200, 250, or 300 ng·h/ml. In some embodiments, trametinib is administered at a dose that provides an area under the CSF concentration curve of trametinib of about 20 to about 700 ng·h/ml, about 20 to about 600 ng·h/ml, about 30 to about 500 ng·h/ml, about 50 to about 400 ng·h/ml, about 50 to about 300 ng·h/ml, about 50 to about 200 ng·h/ml, about 50 to about 100 ng·h/ml, about 60 to 300 ng·h/ml, about 30 to about 200 ng·h/ml.

In some embodiments, trametinib is administered at a dose that provides a mean peak trametinib concentration (Cₘₐₓ) of at least 0.25 ng/ml in the plasma. In some embodiments, trametinib is administered at a dose that provides a mean peak trametinib concentration (Cₘₐₓ) of at least 0.5, 0.75, 1, 1.25, 1.50, 1.75, 2, 2.25, 2.50, 2.75, 3, 3.25, 3.50, 3.75, 4, 4.25, 4.50, 4.75, or 5 ng/ml in the plasma. In some embodiments, trametinib is administered at a dose that provides a mean peak trametinib concentration (Cₘₐₓ) of at least 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 or 6.0 ng/ml in the plasma. In some embodiments, trametinib is administered at a dose that provides a mean peak trametinib concentration (Cₘₐₓ) of about 4, 5, 6, 7, 8, 9, 10, 11, 12, 13, 14, 15, 16, 17, 18, 19, 20, 21, 22, 23, 24, 25, 26, 27, 28, 29, or 30 ng/ml in the plasma. In some embodiments, trametinib is administered at a dose that provides a mean peak trametinib concentration (Cₘₐₓ) of between 1 and 200, between 1 and 150, between 1 and 100, between 2 and 100, between 3 and 100, between 4 and 100, between 5 and 100, between 10 and 100, between 15 and 100, between 15 and 90, between 20 and 80, between 2.5 and 50, between 2.5 and 25, between 2.5 and 10 ng/ml, between 3 and 50 ng/ml in the plasma.

In some embodiments, trametinib is administered at a dose that provides an area under the plasma concentration curve of trametinib of at least 25, 30, 35, 40, 45, 50, 55, 60, 65, 70, 75, 80, 85, 90, 95, 100, 110, 120, 130, 140, 150, 160, 170, 180, 190 or 200 ng·h/mL. In some embodiments, trametinib is administered at a dose that provides an area under the plasma concentration curve of trametinib of about 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 400, or 500 ng·h/mL. In some embodiments, trametinib is administered at a dose that provides an area under the plasma concentration curve of trametinib of about 20 to about 700 ng·h/mL, about 20 to about 600 ng·h/mL, about 30 to about 500 ng·h/mL, about 50 to about 400 ng·h/mL, about 50 to about 300 ng·h/mL, about 50 to about 200 ng·h/mL, about 50 to about 100 ng·h/mL, about 100 to about 500 ng·h/mL.

In some embodiments, trametinib is administered at a dose between 0.5 and 2 mg/day. In some embodiments, trametinib is administered at a dose between 0.75 and 2 mg/day. In some embodiments, trametinib is administered at a dose between 1 and 2 mg/day. In some embodiments, trametinib is administered at a dose between 0.75 and 1.25 mg/day. In some embodiments, trametinib is administered at a dose between 0.5 and 1 mg/day. In some embodiments, trametinib is administered at a dose of 0.5 mg/day. In some embodiments, trametinib is administered at a dose of 1 mg/day. In some embodiments, trametinib is administered at a dose of 1.5 mg/day. In some embodiments, trametinib is administered at a dose of 2 mg/day.

In some embodiments, trametinib is administered at a dose greater than 0.5 mg/day and lower than 2 mg/day. In some embodiments, trametinib is administered at a dose greater than 0.75 mg/day and lower than 2 mg/day. In some embodiments, trametinib is administered at a dose greater than 1 mg/day and lower than 2 mg/day. In some embodiments, trametinib is administered at a dose greater than 0.75 mg/day and lower than 1.25 mg/day. In some embodiments, trametinib is administered at a dose greater than 0.5 and lower than 1 mg/day.

In a preferred embodiment, each dose is a daily dose delivered as a single oral uptake. In some embodiments, each dose is divided into several oral uptakes. In some embodiments, each dose is divided into equal uptake doses. In some embodiments, each dose is divided into unequal uptake doses. In preferred embodiments, each dose is administered at regular intervals.

### 4. Detection of Markers

In another aspect, a method of testing the therapeutic outcome of a drug (e.g., MEK 1/2 inhibitor such as trametinib) in a neurodegenerative subject is provided. The method involves the step of measuring the level of one or more markers in a sample obtained from the subject.

In some embodiments, the method provided herein further comprises the step of testing the expression of one or more markers in a sample obtained from the subject. Expression of one or more markers can be tested using a method known in the art by measuring proteins or by measuring mRNAs, using methods such as western blotting, ELISA, RT-PCR, qPCR, immunoelectrophoresis, protein immunoprecipitation, and protein immunostaining. Various methods of measuring amounts of mRNA or proteins can be adopted for the method.

In some embodiments, the method provided herein further comprises the step of measuring the level of one or more marker proteins in a sample obtained from the subject. Level of one or more marker proteins can be measured using various protein assays known in the art. For example, the sample may be contacted with an antibody specific for said marker under conditions sufficient for an antibody-marker complex to form, and then detecting said complex. The presence of the protein biomarker may be detected in a number of ways, such as western blotting, ELISA, immunoelectrophoresis, protein immunoprecipitation, protein immunostaining, 2-dimensional SDS-PAGE, fluorescence activated cell sorting (FACS), and flow cytometry.

The level of one or more markers can be measured at multiple time points, and the amounts measured at different time points can be compared. Changes in the level of one or more markers over time can be used to determine the therapeutic effects of an MEK 1/2 inhibitor such as trametinib in the patient.

In some embodiments, each of the one or more markers is encoded by a human homolog of the mouse gene selected from the group consisting of: Gabrb1, Gabrr2, Glra3, Nr3c2, Cdk15, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Crc15, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Den, and Hmgb1.

In some embodiments, each of the one or more markers is a protein related to lysosomal activity. The protein related to lysosomal activity can be glycohydrolase or protease. The glycohydrolase can be selected from the group consisting of: β-hexosaminidase, β-galactosidase, β-galactosylcerebrosidase, β-glucuronidase. In some embodiments, the protease can be cathepsin. In some embodiments, the cathepsin can be selected from the group consisting of: Cathepsin S, Cathepsin D, Cathepsin B, Cathepsin K, and Cathepsin L.

In some embodiments, the level of one or more markers previously known to be associated with a neurodegenerative disease are measured.

In some embodiments, one or more markers are selected from the group consisting of (1) markers previously known to be associated with a neurogenerative disease (e.g., AD); (2) a protein or mRNA encoded by a human homolog of the mouse gene selected from the group consisting of Gabrb1, Gabrr2, Glra3, Nr3c2, Cdkl5, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez), Atp6v0c, Rnase6, Ctsk, Acr, Prss16, Lamp5, Prdx6, Unc3d, Bag3, Tial1, Adrb2, Hps4, Ass1, Cckar, Gimap5, Hmox1, Sesn3, Pcsk9, Capn1, Rnf152, Vps13c, Den, and Hmgb1; and (3) a protein related to lysosomal activity, such as glycohydrolase or protease. The glycohydrolase can be selected from the group consisting of: β-hexosaminidase, β-galactosidase, β-galactosylcerebrosidase, β-glucuronidase. In some embodiments, the protease can be cathepsin. In some embodiments, the cathepsin can be selected from the group consisting of: Cathepsin S, Cathepsin D, Cathepsin B, Cathepsin K, and Cathepsin L.

In some embodiments, the level of one or more markers is measured in a sample obtained after the step of commencing administration of trametinib. The sample can be obtained at one or multiple time points after the step of commencing administration of trametinib. For example, the sample is obtained 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks after commencing administration of trametinib. In some embodiments, the sample is obtained 1 month, 2 months, 3 months, 4 months, 5 months, or 6 months after commencing administration of trametinib. In some embodiments, the sample is obtained at once after the step of commencing administration of trametinib. In some embodiments, the sample is obtained at 2 different time points after the step of commencing administration of trametinib. In some embodiments, the sample is obtained at 3 different time points after commencing administration of trametinib. In some embodiments, the sample is obtained at 4, 5, 6, 7, or 8 different time points after the step of commencing administration of trametinib.

In some embodiments, the level of one or more markers is measured in a control sample obtained before commencing administration of trametinib. In some embodiments, the level of one or more markers is measured in a biological sample obtained from the healthy subjects who are free of the disease(s) of interest. In some embodiments, the method further comprises the step of comparing the level of one or more markers in the control sample obtained before commencing administration of trametinib to samples obtained after administration of trametinib. In some embodiments, the method further comprises the step of comparing the level of one or more markers in the healthy subjects who are free of the disease(s) of interest to the level in the samples obtained from patients before commencing administration or after administration of trametinib. Comparison of level of one or more markers can be used to determine therapeutic effects of trametinib. In some embodiments, the level of one or more markers can be used to determine appropriate duration or dose of trametinib administration to achieve desired therapeutic outcome. In some embodiments, time-course analysis of the one or more markers is performed. In some embodiments, the level of one or more markers can be used to determine the methods of subsequent trametinib administration, such as duration and dose of trametinib. In some embodiments, the level of one or more markers can be used to identify individuals who are more likely than similar individuals without the biomarker to experience a favorable effect from exposure to trametinib.

The sample used for testing markers can be obtained by any of the methods known in the art. For example, the sample can be obtained by brain biopsy. In some embodiments, the sample is obtained by stereotactic brain biopsy. In some embodiments, the sample is obtained from body fluids or secretions of a patient, such as blood, cerebrospinal fluid (CSF), urine, body secreting fluid, saliva, stool, pleural fluid, lymphatic fluid, sputum, ascites, prostatic fluid, or any other bodily secretion or derivative thereof. Blood sample includes whole blood, plasma, serum, peripheral blood mononuclear cells (PBMC), or any components of blood.

In another aspect, a composition for use in determining therapeutic effect of a MEK 1/2 inhibitor such as trametinib, comprising a probe for specifically detecting a marker is presented. In another aspect, kits for such purpose are also provided. Such kits may comprise a carrier being compartmentalized to receive in close confinement one or more containers such as vials, tubes, and the like, each of the containers comprising one of the separate elements to be used in the method. For example, one of the containers may comprise a probe that is or can be detectably labeled. Such probe may be an antibody or polynucleotide specific for a protein or mRNA, respectively. Such kit will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific application and may also indicate directions for either in vivo or in vitro use, such as those described above.

A typical embodiment is a kit comprising a container, a label on said container, and a composition contained within said container, wherein the composition includes a primary antibody that binds to a protein or autoantibody biomarker, and the label on said container indicates that the composition can be used to evaluate the presence of such proteins or antibodies in a sample, and wherein the kit includes instructions for using the antibody for evaluating the presence of biomarker proteins in a particular sample type. The kit can further comprise a set of instructions and materials for preparing a sample and applying antibody to the sample. The kit may include both a primary and secondary antibody, wherein the secondary antibody is conjugated to a label.

### 5. Pharmaceutical Compositions and Unit Dosage Form

In yet another aspect, the present disclosure provides a pharmaceutical composition and a unit dosage form comprising trametinib for treatment of a neurodegenerative disease (e.g., AD).

In typical embodiments, trametinib is formulated for oral administration. In some embodiments, trametinib is formulation with an inert diluent or with an edible carrier. In various embodiments, trametinib is enclosed in hard or soft shell gelatin capsules, compressed into tablets, or incorporated directly into the food of the diet. For oral therapeutic administration, the active compound may be incorporated with an excipient and used in the form of ingestible tablets, buccal tablets, coated tablets, troches, capsules, elixirs, dispersions, suspensions, solutions, syrups, wafers, patches, powder for oral solution and the like.

Tablets, troches, pills, capsules and the like may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the unit dosage form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for the material in a dosage form or pharmaceutical composition to be pharmaceutically pure and substantially non-toxic in the amounts employed.

Some compositions or dosage forms may be a liquid, or may comprise a solid phase dispersed in a liquid.

In some embodiments, an oral dosage form may comprise a silicified microcrystalline cellulose such as PROSOLV®. For example, about 20% (wt/wt) to about 70% (wt/wt), about 10% (wt/wt) to about 20% (wt/wt), about 20% (wt/wt) to about 40% (wt/wt), about 25% (wt/wt) to about 30% (wt/wt), about 40% (wt/wt) to about 50% (wt/wt), or about 45% (wt/wt) to about 50% (wt/wt) silicified microcrystalline cellulose may be present in an oral dosage form or a unit of an oral dosage form.

In some embodiments, an oral dosage form may comprise a crosslinked polyvinylpyrrolidone such as crospovidone. For example, about 1% (wt/wt) to about 10% (wt/wt), about 1% (wt/wt) to about 5% (wt/wt), or about 1% (wt/wt) to about 3% (wt/wt) crosslinked polyvinylpyrrolidone may be present in an oral dosage form or a unit of an oral dosage form.

In some embodiments, an oral dosage form may comprise a fumed silica such as AEROSIL® For example, about 0.1% (wt/wt) to about 10% (wt/wt), about 0.1% (wt/wt) to about 1% (wt/wt), or about 0.4% (wt/wt) to about 0.6% (wt/wt) fumed silica may be present in an oral dosage form or a unit of an oral dosage form. In some embodiments, an oral dosage form may comprise magnesium stearate. For example, about 0.1% (wt/wt) to about 10% (wt/wt), about 0.1% (wt/wt) to about 1% (wt/wt), or about 0.4% (wt/wt) to about 0.6% (wt/wt) magnesium stearate may be present in an oral dosage form or a unit of an oral dosage form. An oral dosage form comprising zoledronic acid or another bisphosphonate may be included in a pharmaceutical product comprising more than one unit of the oral dosage form.

Trametinib may be formulated for other administration methods, for example, sublingual, rectal, intranasal, parenteral, transdermal or local administration, or injections. Solutions of the active compounds as free acids or pharmacologically acceptable salts can be prepared in water suitably mixed with a surfactant, such as hydroxypropylcellulose. A dispersion can also have an oil dispersed within, or dispersed in, glycerol, liquid polyethylene glycols, and mixtures thereof. Under ordinary conditions of storage and use, these preparations may contain a preservative to prevent the growth of microorganisms.

In preferred embodiments, each unit of the oral dosage form contains an effective amount for daily administration. In some embodiments, each unit of the oral dosage form contains between 0.1 and 3 mg of trametinib. In some embodiments, each unit of the oral dosage form contains between 0.2 and 3 mg of trametinib. In some embodiments, each unit of the oral dosage form contains between 0.3 and 3 mg of trametinib. In some embodiments, each unit of the oral dosage form contains between 0.4 and 3 mg of trametinib. In some embodiments, each unit of the oral dosage form contains between 0.5 and 3 mg of trametinib. In some embodiments, each unit of the oral dosage form contains between 0.5 and 2.5 mg of trametinib. In some embodiments, each unit of the oral dosage form contains between 0.5 and 2 mg of trametinib. In some embodiments, each unit of the oral dosage form contains between 0.75 and 2.5 mg of trametinib. In some embodiments, each unit of the oral dosage form contains between 1 and 2 mg of trametinib. In some embodiments, each unit of the oral dosage form contains between 0.75 and 1.25 mg of trametinib. In some embodiments, each unit of the oral dosage form contains 0.2 mg of trametinib. In some embodiments, each unit of the oral dosage form contains 0.25 mg of trametinib. In some embodiments, each unit of the oral dosage form contains 0.5 mg of trametinib. In some embodiments, each unit of the oral dosage form contains 1 mg of trametinib. In some embodiments, each unit of the oral dosage form contains 1.5 mg of trametinib. In some embodiments, each unit of the oral dosage form contains 2 mg of trametinib. In some embodiments, each unit of the oral dosage form contains 2.5 mg of trametinib. In some embodiments, each unit of the oral dosage form contains 3 mg of trametinib.

In some embodiments, each unit of the oral dosage form is a MEKINIST tablet containing 0.5 mg, 1 mg, or 2 mg of trametinib. In some embodiments, each 0.5 mg tablet contains 0.5635 mg trametinib dimethyl sulfoxide equivalent to 0.5 mg of trametinib nonsolvated parent. In some embodiments, each 1 mg tablet contains 1.127 mg trametinib dimethyl sulfoxide equivalent to 1 mg of trametinib non-solvated parent. In some embodiments, each 2 mg tablet contains 2.254 mg trametinib dimethyl sulfoxide equivalent to 1 mg of trametinib non-solvated parent.

In some embodiments, the tablet contains from about 25% to about 89% by weight of one or more excipients. In some embodiments, the excipients are substantially free of water. The one or more excipients can be selected from the group consisting of microcrystalline cellulose, powdered cellulose, pregelatinized starch, starch, lactose, Dicalcium phosphate, lactitol, mannitol, sorbitol and maltodextrin. In some embodiments, the amount of unsolvated trametinib does not exceed about 20%. Pharmaceutical composition described in U.S. Pat. Nos. 8,580,304 and 9,271,941, incorporated by reference in their entireties, can be used for various embodiments of the present disclosure.

The tablet can further comprise a tablet core, containing colloidal silicon dioxide, croscarmellose sodium, hypromellose, magnesium stearate (vegetable source), mannitol, microcrystalline cellulose, and sodium lauryl sulfate. The tablet can further comprise a coating containing hypromellose, iron oxide red, iron oxide yellow, polyethylene glycol, polysorbate 80, and/or titanium dioxide.

### 6. Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art.

### 6.1. Example 1: Crossing of Blood-Brain Barrier

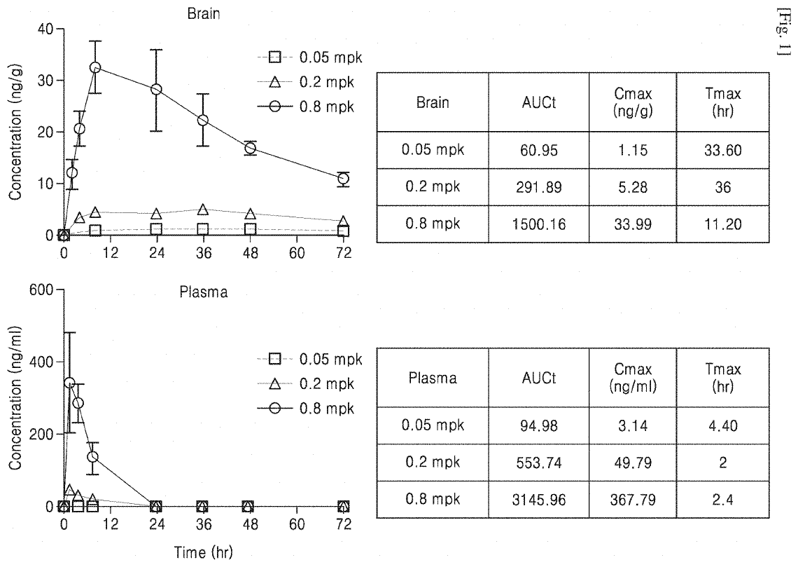

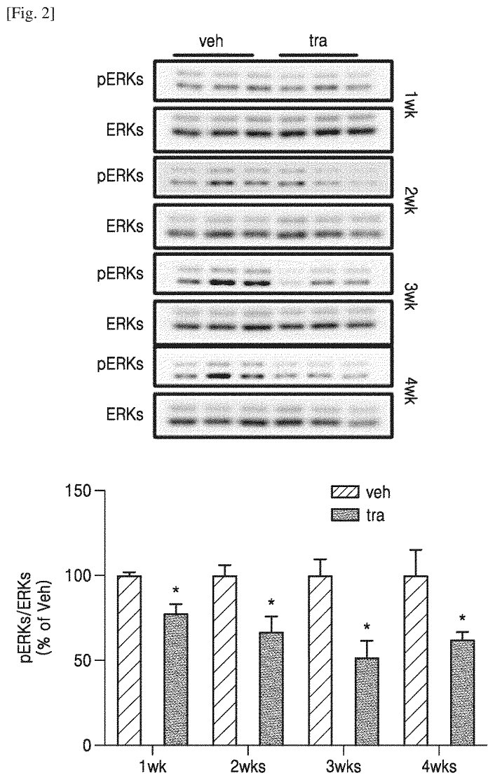

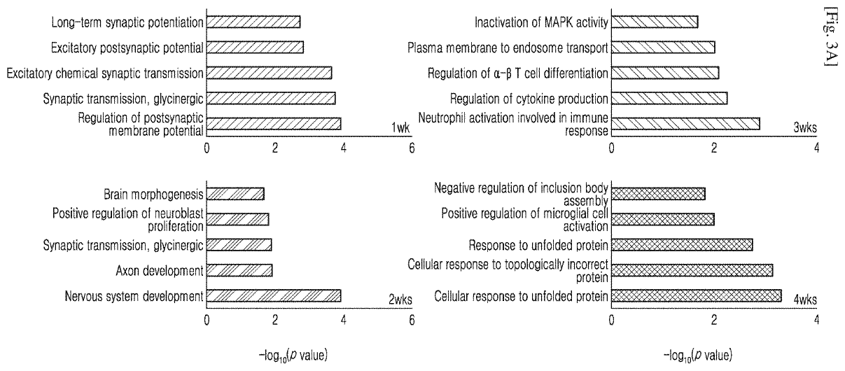

Trametinib was confirmed to penetrate the blood-brain barrier (BBB) after a single oral administration to normal mice. The brain/plasma exposure ratio (AUC) was 47.7% in the highest dose group (FIG. 1). Trametinib was also found to exert its MEK1/2 inhibition in the brains of normal mice after oral administration by causing significant decrease in pERK expression (FIG. 2). The p-ERKs/ERKs ratio decreased by 22.5%, 33.7%, 50% and 45.6% by 1, 2, 3, and 4 week-administrations, respectively, compared to that in the vehicle treated group. These results suggest that trametinib penetrates the BBB.

### 6.2. Example 2: Time-Course of Gene Expression Changes in the Brain after Administration of Trametinib

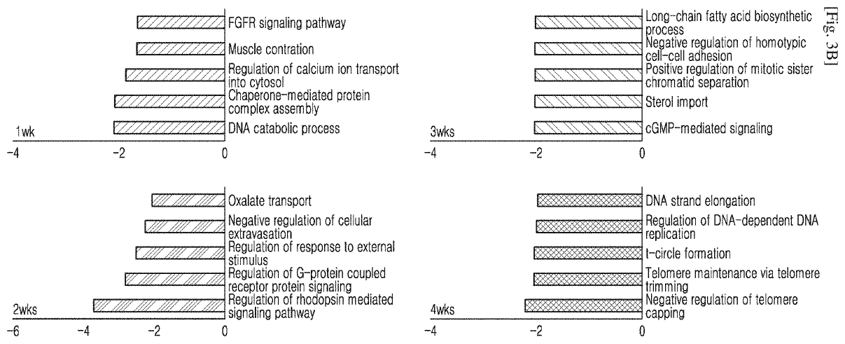

To evaluate the transcriptional profiles in the brain, bulk RNA-Seq was performed using whole brains of normal mice following oral administration of trametinib. Gene ontology terms were enriched at each weekly time point, indicating the relevance of cellular function such as synaptic potential, nervous system development, immune response, and incorrect protein folding in a temporal pattern (FIG. 3A). Decrease in MEK-ERK signaling with trametinib administration during week 1 and week 2 of administration can be indirectly confirmed through the decrease in expression of FGF receptor signaling and GPCR signaling related genes. Decrease in expression of telomere related genes seen in week 4 of trametinib administration (FIG. 3B) suggests it is closely related to neuronal maturation or terminally differentiated neurons through the activation of neurogenesis.

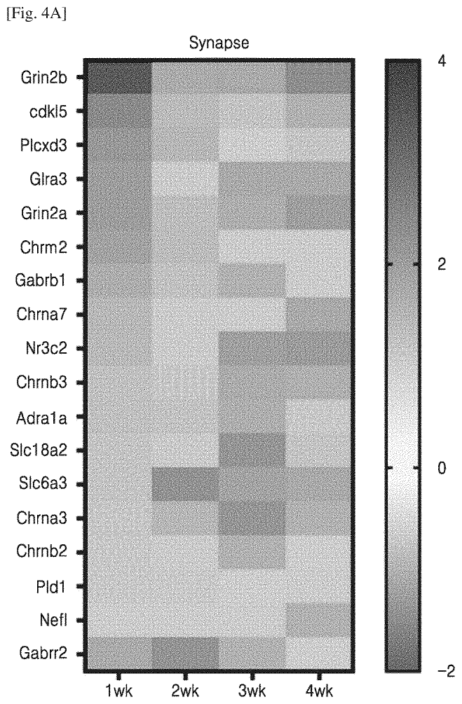

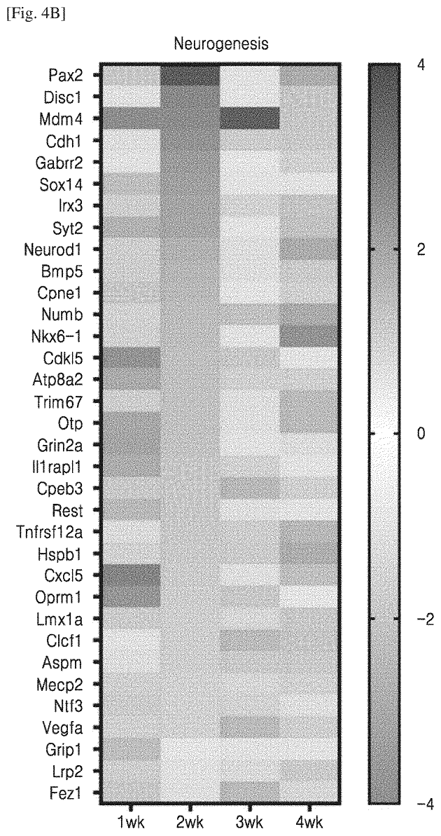

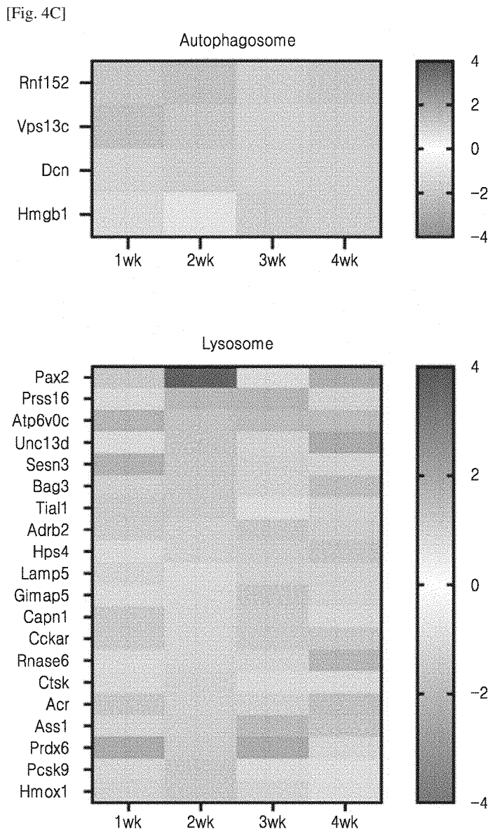

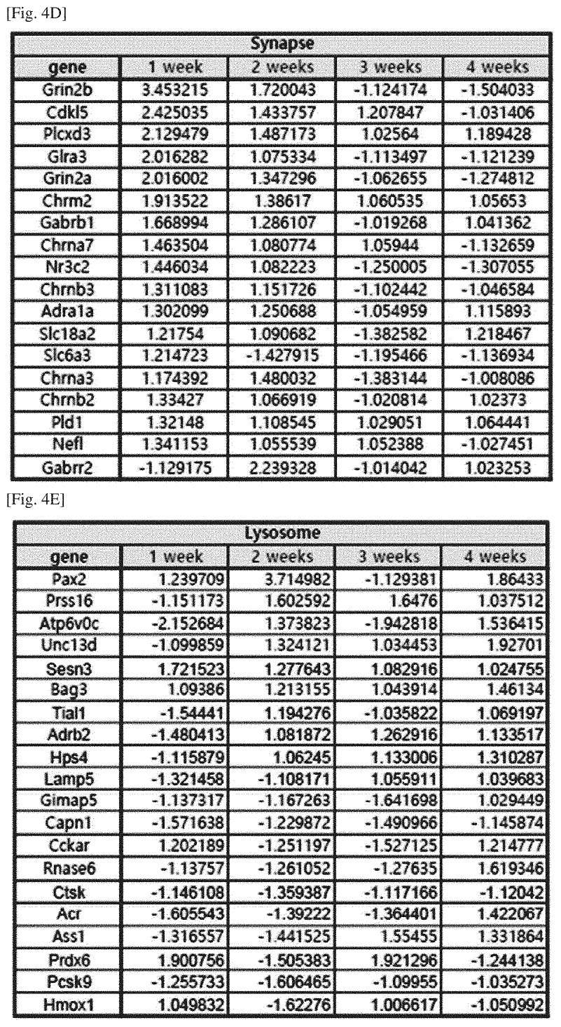

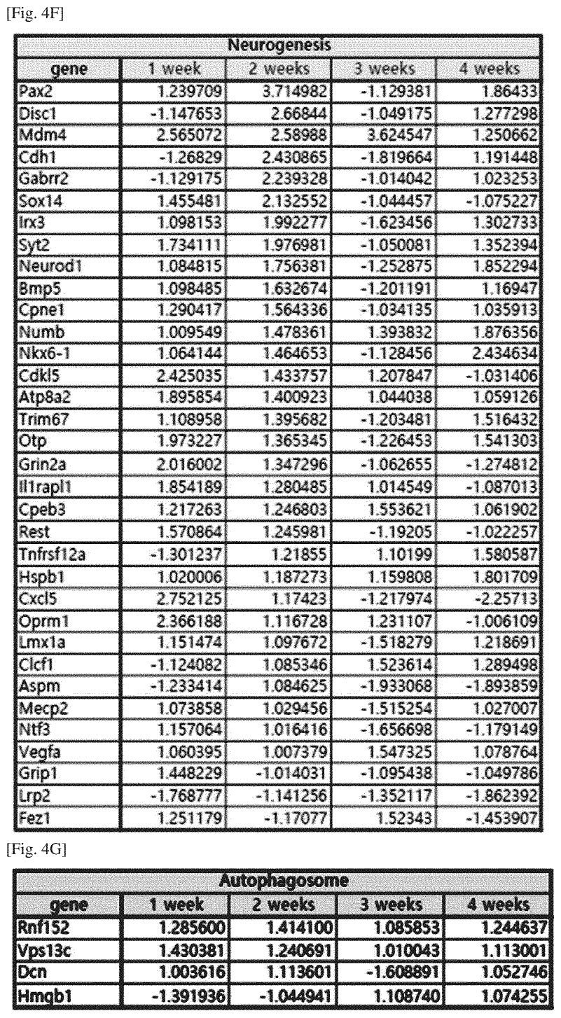

Our result demonstrates that the first week of trametinib administration appears to be the critical period for building neuronal communications as evidenced by the transcriptional changes for synapse formation. In the second week, we observed neuroblast proliferation-related and axon growth-related gene expression, followed by expression of genes for immune reaction and incorrect protein modification reaction in the third and fourth week, respectively. It is important to note that increase in the gene-sets associated with neurogenesis and the induction of lysosomal activity both occurred within the four-week period, as illustrated by the gene expression heatmap (FIGS. 4A-G). Genes set forth in FIGS. 4A-G are those that showed an absolute value of fold change (FC) of at least 1.3 between the vehicle-treated group and trametinib-treated group in at least one of the 1, 2, 3 or 4 week-administration. Their FCs were between −2.26 and 3.71.

The proteins or mRNA encoded by a human homolog of the genes indicated in FIGS. 4A-G or neurotransmitters relating to the protein receptors (GABA, glutamate, acetylcholine, monoamines such as dopamine, and the like) may be used as biomarkers for determining whether a beneficial effect has occurred in an individual who has been exposed to trametinib for the treatment of a neurodegenerative disease. The genes are Gabrb1, Gabrr2, Glra3, Nr3c2, Cdkl5, Grin2a, Grin2b, Plcxd3, Chrm2, Chrna3, Chrna7, Chrnb2, Ne/1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez), Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Dcn, and Hmgb1. The genes are related to synapse formation, neurogenesis, lysosomal function and/or autophagosome.

### 6.3. Example 3: Time-Course of Functional Changes in the Brain after Administration of Trametinib

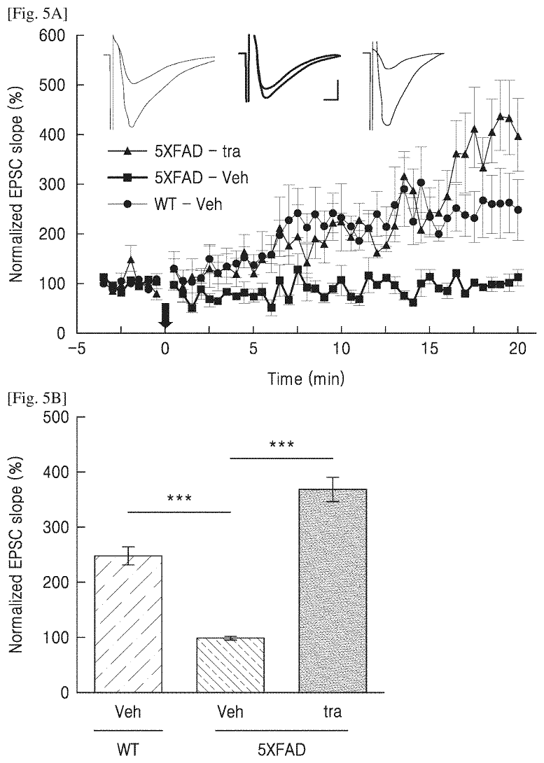

To validate the functional recovery of neural networks, we tested neural activity in the cortex of 5XFAD mice. Cognitive dysfunction in AD has been well correlated with cortical atrophy and memory deficits arising from neuronal loss and breakdown of neural networks. The elaborate interaction between the hippocampal formation (HPF) and the corresponding cortical areas is responsible for information transfer and consolidation. To examine the capability of memory formation in the hippocampus, excitatory postsynaptic currents (EPSCs) were recorded at the hippocampal CA1 region to compare the long-term potentiation (LTP) between wild type and 5XFAD mice at the age of 8 months. The significant reduction in LTP of 5XFAD mice was recovered in the trametinib-orally administered animals to a level almost comparable to that of the wild type control (FIGS. 5A-B).

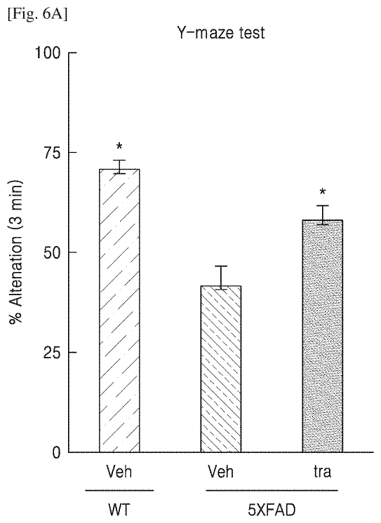

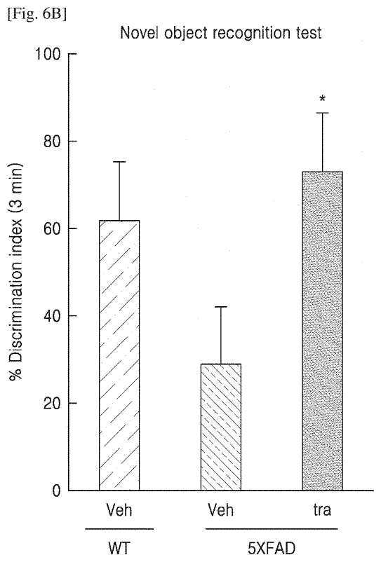

This was consistent with the results from the behavioral studies using Y-maze and novel object recognition tests, which confirmed the recovery of memory formation in trametinib-administered mice (FIGS. 6A-B).

### 6.4. Example 4: Structural Changes in the Brain after Administration of Trametinib

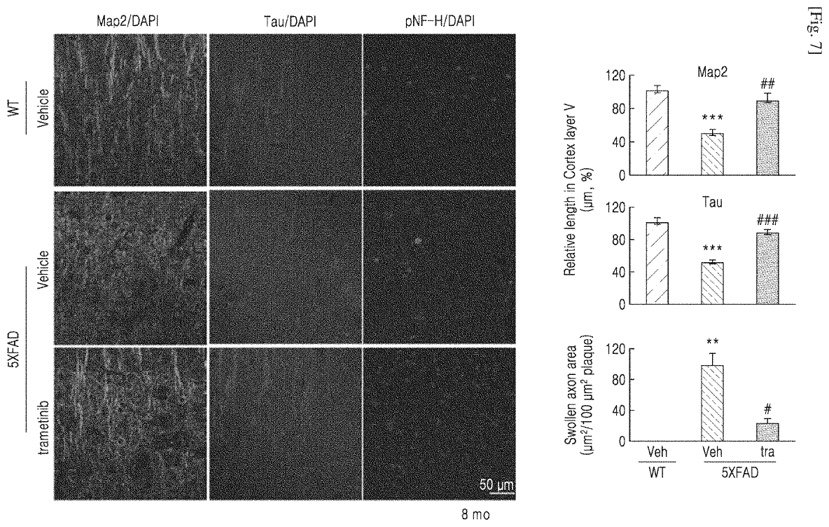

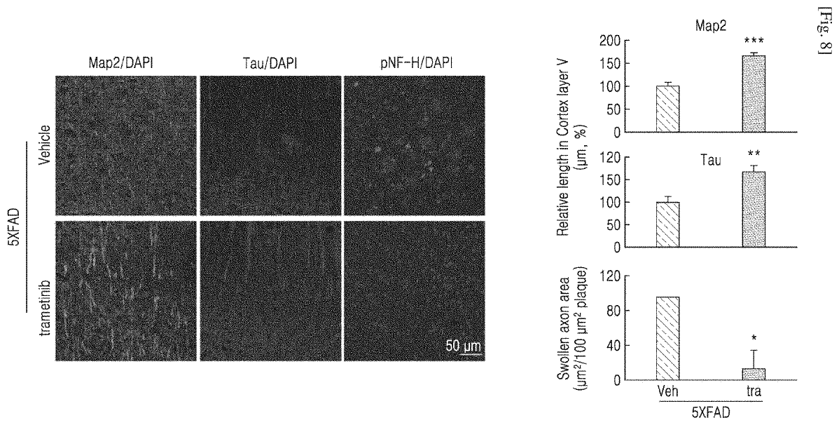

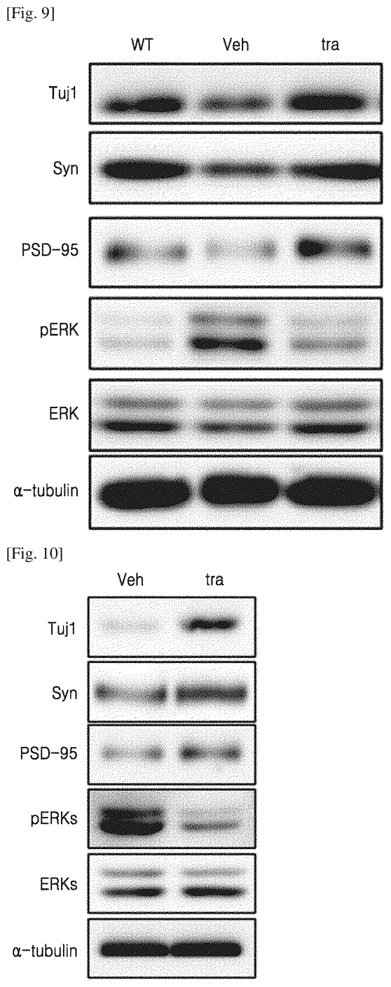

To determine if the functional recovery of the brain by trametinib is due to the structural recovery of neurons, we examined the morphological restoration of axons and dendrites, the critical components comprising the neuronal network. Staining of dendritic and axonal markers Map2 and Tau showed that trametinib treatment led to recovery of dendritic and axonal lengths in the cortex of 8-month-old and 13-month-old 5XFAD mice, whereas those of the vehicle-administered 5XFAD mice showed malformation with shortened lengths. In addition, bulb-like swollen axons, an indicator of axonal deterioration due to AP plaque accumulation in the brains of AD patients and aged monkeys, were also significantly reduced in the trametinib-administered group compared to the vehicle-administered group (FIGS. 7 and 8). Increased expression of the presynaptic marker synaptophysin and postsynaptic marker PSD-95 also revealed the contribution of trametinib in the recovery of synaptic junctions (FIGS. 9 and 10). These results demonstrate that trametinib induces formation of neuronal synapses in addition to conferring neuroprotection by reinforcement of axons and dendrites against amyloid plaque toxicity.

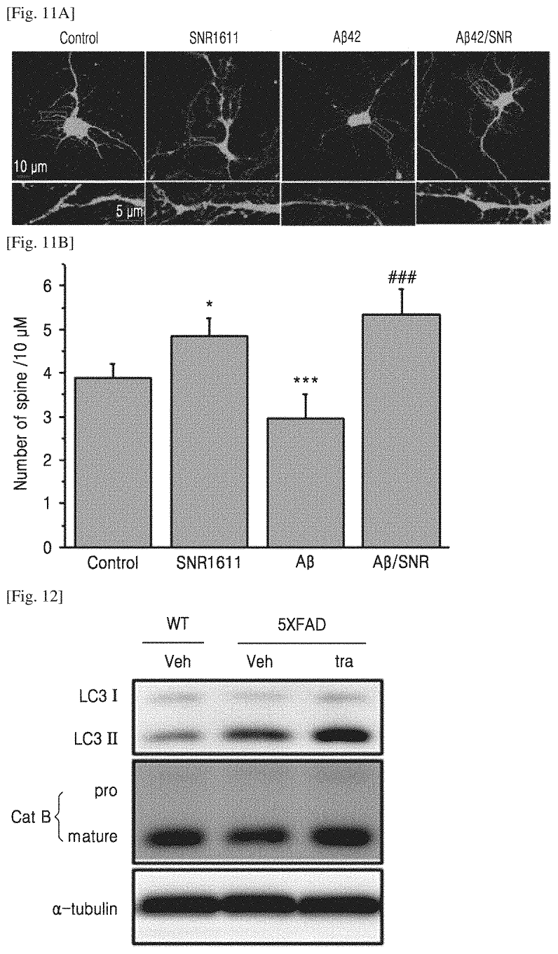

We also examined trametinib's effect on synapse formation in primary cortical neurons from ICR mice embryos by examining the change in dendritic spine formation. Dendritic spines are small protrusions that are present in large numbers on the surface of dendrites. They are postsynaptic components of most excitatory synapses. The number, size and shape of dendritic spines determine neuronal function and provide the structural basis for synaptic plasticity. Trametinib (100 nM) increased the number of dendritic spines by 24% compared to the vehicle treated group. Under Aβ₁₋₄₂ oligomer-induced neurotoxic conditions, the number of dendritic spines decreased by 24% compared to the vehicle treated group (CTL), while the number increased by 80% trametinib treatment compared to the Aβ₁₋₄₂ treated alone group (FIGS. 11A and 11B). The increase in dendritic spine number by trametinib indicates that synapse dysfunction induced by Aβ₁₋₄₂ was recovered by trametinib.

### 6.5. Example 5: Enhancement of Lysosomal Activity by Trametinib Through Autophagosome-Lysosome Fusion

We examined the possibility of a decrease in apoptosis and enhancement in lysosomal activity by trametinib in cortex layer V of the 5XFAD mice brain. Enhancement in autophagic lysosomal activity was confirmed by several markers in 8-month old 5XFAD mice brain (FIG. 12). The autophagosome marker, LC3-II, increased in the vehicle-administered group and further increased in the trametinib-administered group. The level of mature cathepsin B, one of the lysosomal proteases, decreased in the vehicle-administered 5XFAD mice. In contrast, the level of mature cathepsin B was seen to increase in the trametinib-administered group, indicating that lysosomal degradation of neurotoxic proteins may be induced by trametinib (FIG. 12).

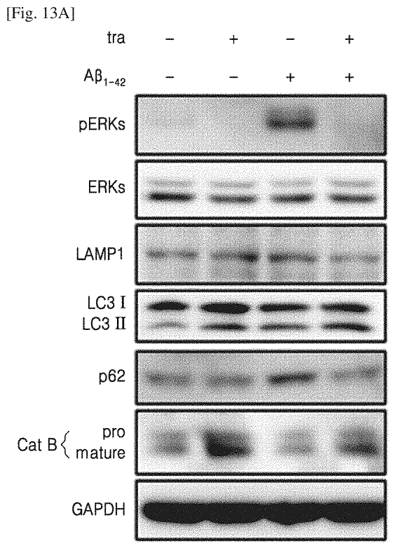

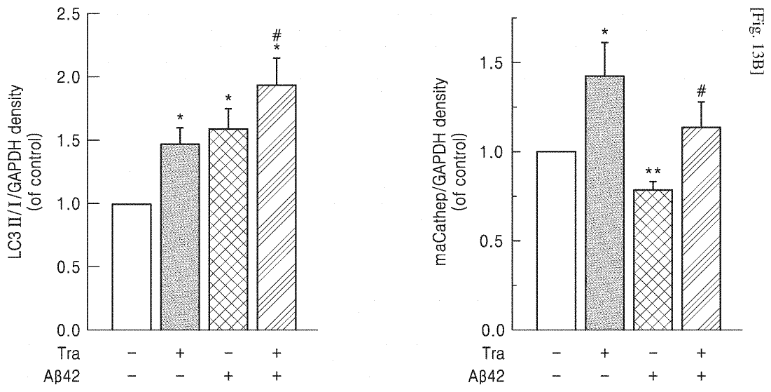

The ability of trametinib to induce autophagic lysosomal activity was also confirmed in the neuronal cell line (SH-SY5Y) (FIGS. 13A-B). Similar to the results obtained with 5XFAD mice, pERK and LC3-II levels increased in SH-SY5Y cells treated with Aβ₁₋₄₂ oligomers. When the Aβ₁₋₄₂ cells were treated with trametinib, the level of LC3-II/LC-I increased by 65% compared to Aβ₁₋₄₂ treated alone cells, and the level of mature cathepsin B increased by 44% compared to Aβ₁₋₄₂ treated alone cells (FIG. 13B). Degradation of p62 as a marker of autophagic flux was observed with trametinib treatment even in the presence of Aβ₁₋₄₂ oligomers (FIG. 13A). Similar results were observed in primary cortical neurons (FIG. 14A). Particularly, the level of mature cathepsin B increased by 54% with treatment compared to the vehicle treated control (CTL). When primary cortical neurons were treated with Aβ₁₋₄₂ oligomers, the level of mature cathepsin B decreased by 26% compared to the non-treated neurons (CTL), but the level increased by 73% with trametinib treatment compared to the Aβ₁₋₄₂ treated alone group (FIG. 14B).

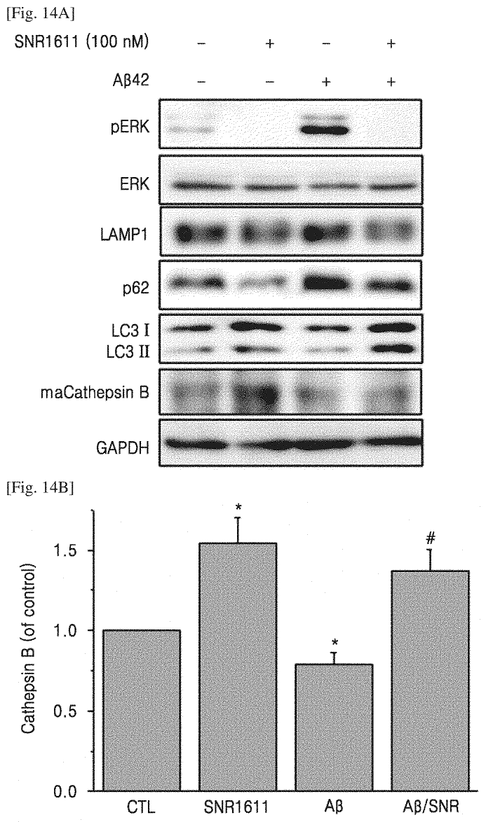

We then performed immunocytochemical analysis to confirm the induction of lysosomal activity by autophagosome-lysosome fusion in SH-SY5Y cells. We found that cells treated with trametinib showed increased co-staining of LC3-LAMP1 even in the presence of Aβ₁₋₄₂ oligomers (FIGS. 15A-B). To assess lysosomal acidification, we measured lysotracker-positive acidic puncta. Trametinib treatment resulted in increased lysotracker-positive acidic puncta in the presence of Aβ₁₋₄₂ oligomers (FIGS. 15A-B).

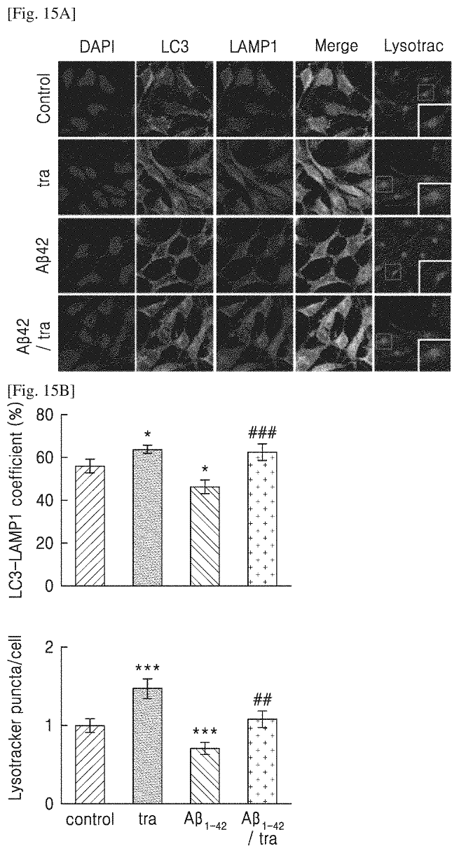

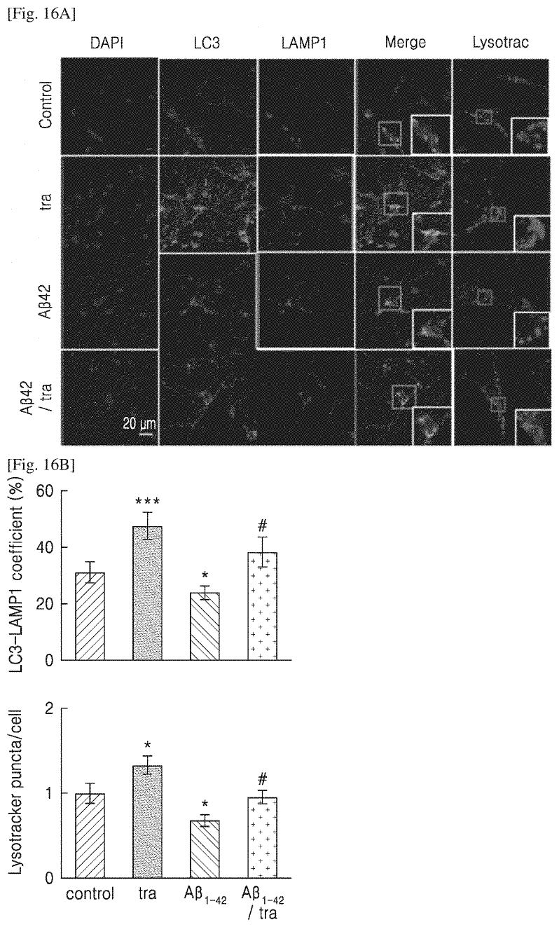

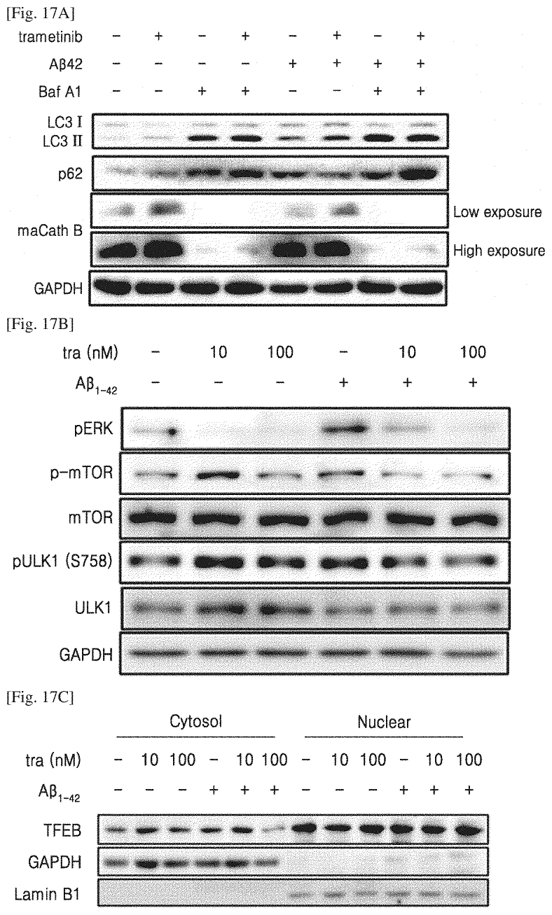

We also investigated these effects in primary cortical neurons. Neurons treated with trametinib showed increased co-staining of LC3-LAMP1 and lysotracker-positive acidic puncta even in the presence of Aβ₁₋₄₂ oligomers (FIGS. 16A-B). These results imply that trametinib activates lysosomal degradation of neurotoxins by inducing the autophagic-lysosomal fusion. Further confirmation of trametinib's involvement in autophagosome-lysosome fusion was provided by the use of bafilomycin A1 that interrupts the fusion of autophagosomes and lysosomes by inhibiting V-ATPase. Treatment with trametinib and bafilomycin A1 eliminated the effect of trametinib on increasing mature cathepsin B and decreasing p62 in the presence of Aβ₁₋₄₂ oligomers (FIG. 17A).

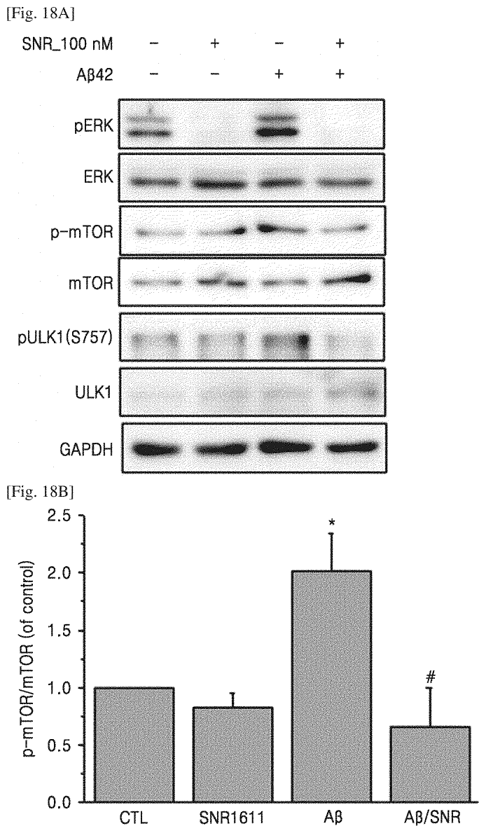

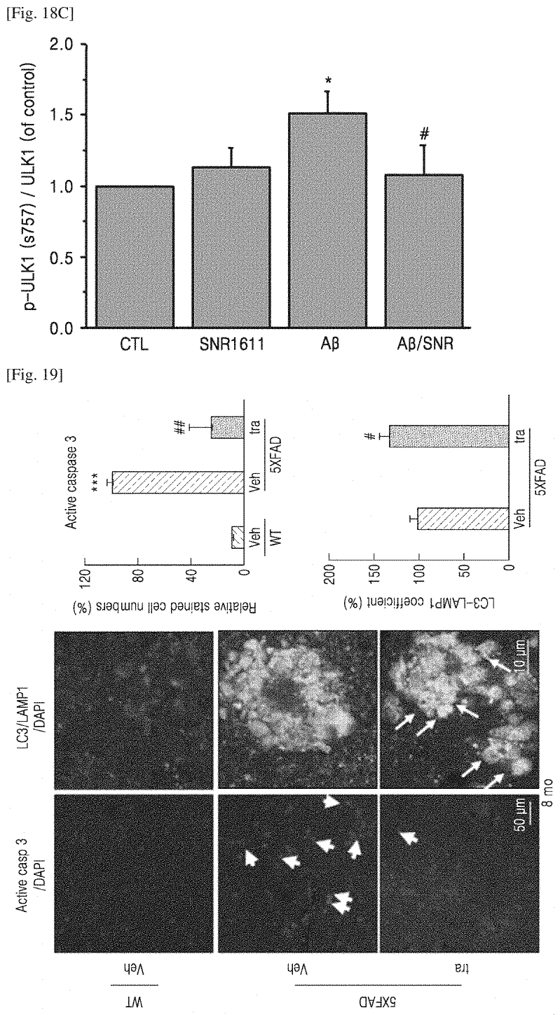

To examine the mechanism by which trametinib triggers autophagic flux, changes in the downstream mediators of autophagy were measured using western blotting in SH-SY5Y cells. Inactivation of mTOR leads to dephosphorylation of ULK1 on Ser758 (in human, Ser757 in mice) and subsequent autophagy induction. Indeed, we observed that trametinib inhibited the phosphorylation of mTOR and ULK1 on Ser758 in SH-SY5Y cells in the presence of Aβ₁₋₄₂ oligomers (FIG. 17B). Similar results were observed in primary cortical neurons treated with Aβ₁₋₄₂ oligomers (FIG. 18A-C). p-mTOR/mTOR ratio increased 2-fold by Aβ₁₋₄₂ treatment compared to non-treated group. In the group co-treated with trametinib (100 nM) and Aβ₁₋₄₂, the ratio decreased by 67% compared to the Aβ₁₋₄₂ treated alone group (FIG. 18B). Furthermore, the ratio of ULK1 phosphorylated on Ser757 (pULK1(S757)) to total ULK1 increased by 51% in the Aβ₁₋₄₂ treated neurons compared to non-treated neurons, while the ratio decreased by 29% in the neurons co-treated with trametinib and Aβ₁₋₄₂ compared to the Aβ₁₋₄₂ treated alone neurons (FIG. 18C). We also tested whether trametinib affects the translocation of TFEB, a transcription factor EB that regulates lysosomal genes and autophagy-related genes. ERK2 and mTORC1 can phosphorylate TFEB on Ser142, resulting in inhibition of translocation from the cytosol to the nucleus and prevention of transcription of lysosomal and autophagy-related genes. We confirmed that trametinib treatment in the presence of Aβ₁₋₄₂ oligomers induced nuclear translocation of TFEB (FIG. 17C), indicating that trametinib dephosphorylated TFEB and localized it to the nucleus.

In addition to increase in autophagosome-lysosome fusion, reduction of apoptosis was seen in the trametinib-administered group. In order to determine whether the increase in autophagic flux induces a decrease in toxic proteins and leads to reduced apoptosis, we examined the co-staining of LC3-LAMP1 and expression of the apoptosis marker active caspase 3 in the 5XFAD mice cortex. Increased LC3-LAMP1 co-stained cells and reduced apoptosis were seen in the trametinib-administered group (FIG. 19).

Taken together, these findings indicate that trametinib inhibits Aβ₁₋₄₂-induced cell death by facilitating lysosomal activity through increased autophagosome-lysosome fusion and downregulation of the mTOR pathway, meanwhile inducing the differentiation of NSCs into neuronal lineages.

In the presence of toxic Aβ₁₋₄₂ oligomers or under amyloid plaque conditions, it is known that autophagic flux and lysosomal activity are reduced resulting in eventual cell death. In this study, we demonstrated that trametinib recovers autophagic flux and lysosomal activity in the toxic environment via induction of autophagosome-lysosome fusion. As for its mechanism of action, trametinib inhibits mTOR phosphorylation and reduces Ulk1 phosphorylation at Ser 758, which may in turn allows increased interaction of ULK1 with AMPK for autophagic induction. The maintenance of proteostasis through lysosomal activation not only induces protection through neurotoxin removal, but also reverses age-related phenotype (rejuvenation) through intracellular metabolic activation. Accordingly, the induction of lysosomal activation and neurogenesis possibly act synergistically to bring about the recovery effect in the AD patient's brain.

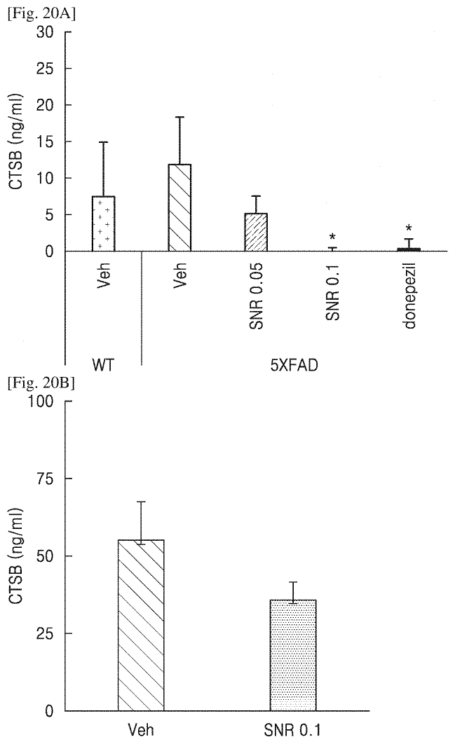

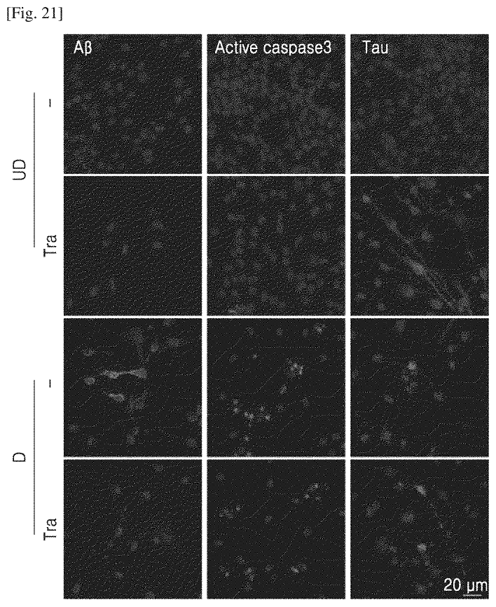

We also examined whether changes in the level of endogenous molecules related to lysosomal activity can be detected in the plasma of the 5XFAD mice treated with trametinib. Plasma cathepsin B level showed a decreasing trend in 8-month-old 5XFAD mice administered with 0.05 and 0.1 mg/kg/day trametinib (SNR0.05, SNR0.1), with the decrease reaching statistical significance in the 0.1 mg/kg/day treated group (decreased by 56.16% in the 0.05 mg/kg/day group and decreased by 99.2% in the 0.1 mg/kg/day group compared to vehicle treated 5XFAD mice group) (FIG. 20A). The donepezil-administered group also exhibited a statistically significant decrease in cathepsin B level in comparison to the 5XFAD-vehicle (decreased by 97.13% compared to vehicle treated 5XFAD mice group) (FIG. 20A). In the 13-month-old 5XFAD mice, plasma cathepsin B level showed a decreasing trend in the 5XFAD mice treated with 0.1 mg/kg/day trametinib (SNR 0.1) compared to the 5XFAD-vehicle group (Veh), although not statistically significant (FIG. 20B).

There are reports of increased plasma cathepsin B levels in persons with Alzheimer's Disease compared to healthy controls (Morena, F. et al. A Comparison of Lysosomal Enzymes Expression Levels in Peripheral Blood of Mild- and Severe-Alzheimer's Disease and MCI Patients: Implications for Regenerative Medicine Approaches. Int J Mol Sci 18, doi:10.3390/ijms18081806 (2017); Sundelof, J. et al. Higher cathepsin B levels in plasma in Alzheimer's disease compared to healthy controls. J Alzheimer's Dis 22, 1223-1230, doi:10.3233/JAD-2010-101023 (2010)).

Our results suggest that endogenous molecules related to lysosomal activity can be used as a biomarker for determining whether trametinib has caused a beneficial effect in individuals suffering from neurodegenerative disease. Such molecules include glycohydrolases such as β-hexosaminidase, β-galactosidase, β-galactosylcerebrosidase, β-glucuronidase and proteases such as cathepsins including Cathepsin S, Cathepsin D, Cathepsin B. Cathepsin K, Cathepsin L.

### 6.6. Example 6: Induction of Axogenesis and Protection of Newly Formed Axons

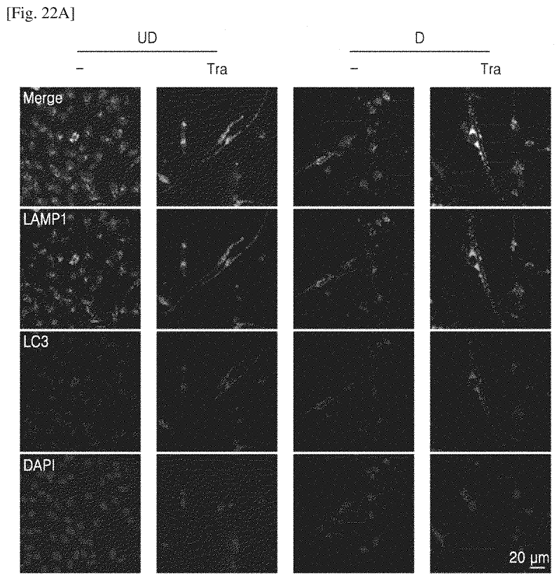

In the NSCs isolated from Tg2576 AD model mice, trametinib reduced active caspase 3 and strongly induced differentiation of NSCs into neuron-like cells (FIG. 21). Tg2576-derived NSCs express human transgenic protein AR and are considered an in vitro AD model resembling some of the cellular alterations observed in vivo (World J Stem Cells 2013; 5(4): 229-237).

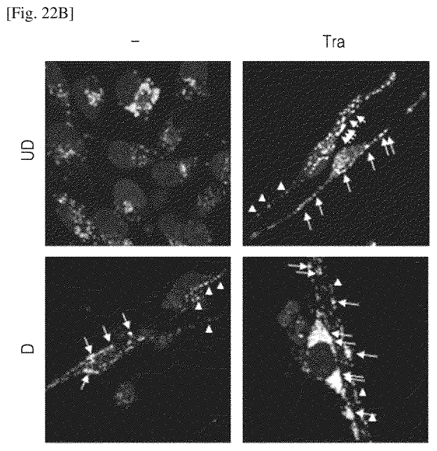

We observed marked increase of cells with bipolar morphology with elongated neurites in the trametinib-treated Tg2576 NSCs (FIG. 21). The expression of autophagosomes and lysosomes markedly increased in the trametinib-treated NSCs, as shown by the increased stainings of LC3 and LAMP1 (FIG. 22A). The autophagosome-lysosome fusions were markedly increased in the axon-like elongated elements as well as the soma of the trametinib-treated NSCs (yellow arrows in FIG. 22B). These results imply that trametinib induces axogenesis and activates lysosomal degradation in the newly formed axons, which shows its potential as a therapeutic agent for diseases associated with axonopathy.

### 6.7. Example 7: Recovery of Myelin Sheaths

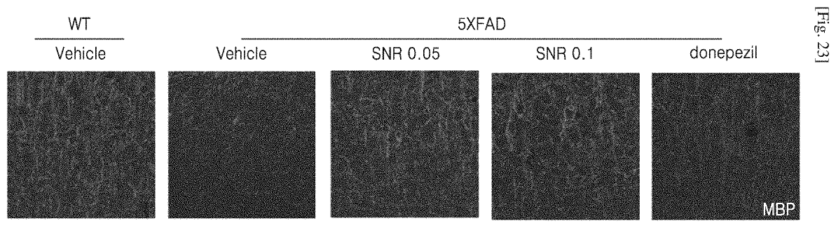

Myelin is an insulating layer that surrounds nerve cell axons and allows electrical impulses to transmit quickly and efficiently along the nerve cells. We examined the effect of trametinib on the myelin sheaths using an antibody against Myelin Basic Protein (MBP), a major constituent of myelin sheaths (FIG. 23). 8-month old 5XFAD mice treated with vehicle showed significant damage in the myelin sheaths compared to the wild type mice of the same age. However, the MBP levels in the 8-month old 5XFAD mice treated with 0.05 mg/kg/day (SNR 0.05) or 0.1 mg/kg/day trametinib (SNR 0.1) were restored back to levels comparable to that of wild type mice. In contrast, the MBP level in the 5XFAD mice treated with donepezil was as low as that of the vehicle treated 5XFAD mice. These results indicate that myelin sheaths damaged in 5XFAD mice can be recovered by trametinib treatment. Recovery of myelin sheaths may enhance the activation of neural cell communication in the brain cortex.

### 6.8. Example 8: Therapeutic Effects of Trametinib in Humans

Trametinib is administered to a patient with Alzheimer's disease in an amount that provides a mean peak trametinib concentration (Cₘₐₓ) of at least 0.25 ng/g in the brain. The administration is performed daily for at least four weeks. Administration of trametinib in this amount and for this period reduces behavioral and/or physiological symptoms associated with Alzheimer's disease.

### 6.9. Experimental Methods

Animals: B6SJL-Tg (APPSwFILon, PSEN1*M146L*L286V) (5XFAD) mice were purchased from The Jackson Laboratory (MMRRC Stock No: 34840-JAX) and experimental procedures were performed according to protocols approved by the Institutional Animal Care and Use Committee (IACUC) of KPCLab (approved number: P171011) or MEDIFRON DBT Inc. (approved number: Medifron 2017-1). C57BL/6 mice were obtained from OrientBio Inc. and compliance with relevant ethical regulations and animal procedures were reviewed and approved by Seoul National University Hospital IACUC (approved number: 16-0043-c1a0) or Yonsei Biomedical Research Institute IACUC (approved number: 2017-0107). ICR mice for pharmacokinetic analysis were obtained from OrientBio Inc. and experimental procedures were approved by IACUC of KPCLab (approved number: P171011).

Trametinib treatment: Trametinib (Medchemexpress, Monmouth Junction, N.J.) was micronized and suspended in the vehicle containing 5% mannitol, 1.5% hydroxypropyl methylcellulose and 0.2% sodium lauryl sulfate. For pharmacokinetic analysis, 0.05, 0.2 and 0.8 mg/kg of trametinib were orally administered to 7-week-old normal mice (ICR, n=5 per group) as a single administration. Mice were sacrificed at each identical time points. For studies on the change in pERK expression in normal mice and whole cell RNA sequencing study, 0.1 mg/kg/day of trametinib (in 4% DMSO+96% corn oil) was orally administered to 6-week old C57BL/6 mice (n=3 per group). Mice were sacrificed after 1, 2, 3, or 4 weeks of administration. 5XFAD mice (male, n=7˜10 per group) were divided into vehicle and trametinib treated groups. 12-month old mice received vehicle or 0.1 mg/kg of trametinib for 1 month by oral gavage once a day (These mice are referred to as “13-month old 5XFAD mice” in the Examples). 5-month old mice received vehicle, 0.05 mg/kg or 0.1 mg/kg of trametinib or 2 mg/kg of donepezil for 3 months by oral gavage once a day (These mice are referred to as “8-month old 5XFAD mice” in the Examples). Blood was obtained after completion of treatment. Blood samples were collected in EDTA tubes which were centrifuged to obtain plasma. All the mice were sacrificed by the perfusion method and brain samples were processed for biochemical and immunohistochemical analysis.

Whole cell RNA sequencing: RNA was isolated from mice whole brain, and cDNA libraries for RNA sequencing were prepared using the TruSeq Stranded mRNA Prep Kit (Illumina, San Diego, Calif.) according to the manufacturer's guidelines (1). The libraries were sequenced on the Illumina Nextseq500 platform, and the reads were mapped to the reference Mouse mm10 genome using Tophat v2.0.13. The total number of reads mapped to the transcriptomes were 24,532, genes and the genes with 0 count in at least one sample were removed before differential expression analysis. There was a total of 18,727 genes after the removal of genes with 0 count. To define differentially expressed genes (DEG), we set up a stringent statistic cutoff of fold change (FC) of ≥1.3 and a false discovery rate (FDR)<0.05. A total of 500 DEGs was identified between the vehicle-treated group and trametinib-treated group in the first week, 498 DEGs in the second week, 446 genes in the third week and 538 genes in the fourth week. Gene ontology was performed with the Gene Ontology program of the gene ontology consortium. Heatmap analysis was performed by R studio using the DEG list related with synapse, neurogenesis and lysosome.

Electrophysiology:

Brain slice preparation: Artificial cerebrospinal fluids (ASCF) were prepared as described by the following components: high sucrose ACSF (mM): 0.5 CaCl₂, 2.5 KCl, 1.25 NaH₂PO₄, 5 MgSO₄, 205 Sucrose, 5 HEPES, 10 Glucose, 26 NaHCO₃ (PH=7.3-7.4, mOsm=300-310), whereas recording ACSF (mM) were prepared as follows: 126 NaCl, 3.5 KCl, 1.25 NaH₂PO₄, 1.6 CaCl₂, 1.2 MgSO₄, 10 Glucose, 26 NaHCO₃, 5 HEPES (PH=7.3-7.4, mOsm=300-310). ACSFs were freshly prepared daily as required.

All experiments were carried out with 8-month old 5XFAD mice. High sucrose ACSF was maintained over ice and saturated by gas infusion of 95% O₂/5% CO₂ for at least 20 mins. Animals were euthanized by carbon dioxide, and the brain was harvested quickly in less than 4 mins and chilled for 2 mins in pre-oxygenated high sucrose ASCF. To make slices, brain hemispheres were sagittally divided. For each hemisphere, cortex and hippocampal sections were coronally sectioned to 300 μm by VF-200 vibratome (Precisionary instrument, USA). For incubation of the slices, they were submerged over nylon mesh in 95% O₂/5% CO₂ oxygenated ASCF for 30 min at 32-34° C., and incubated for an additional 30 mins at room temperature before first recording.

LTP recording with whole-cell Patch clamp: The recording slice was perfused for 30 min in the oxygenated ACSF at 2 ml/min before starting the experiment at 28˜30° C. in the patch clamp chamber. For whole-cell patch clamp we used 4-8 MΩ borosilicate capillary glass electrodes (A-M Systems, USA) pulled from Micropipette puller P1000 (Sutter instrument, USA). The intracellular solution consisted of (in mM): 140 K-gluconate, 10 KCl, 1 EGTA, 10 HEPES, 4 Na₂ATP, 0.3 Na₂GTP in 290 mOsm and pH 7.3 adjusted by KOH. HEKA EPC-10 amplifier double (HEKA Elektronik, Germany) was applied. The slice image was monitored under an upright Eclipse FN1 microscope (Nikon, Japan) through the infrared ray difference interference contrast (IR-DIC) optics with 400× magnification.

An excitatory postsynaptic current (EPSC) was recorded in the voltage clamp mode at −70 mV holding potential in a CA1 pyramidal neuron. The access resistance in the recording cell was below 40 MΩ with marginal 20% tolerance. To stimulate the neuron, a bipolar electrode (FHC, USA) in the external stimulator Iso-flex (A.M.P.I., Israel) was positioned at Schaffer collateral at a distance of 200˜400 μm from the recording electrode. Test stimulation pulses were applied at the same site every 30 seconds with 30-40% intensities from max EPSC amplitude 3 min before the following theta burst stimulation (TBS) for the long-term potentiation (LTP) induction. TBS consisted of four trains with 10 sec intervals, and each train was composed of 5 Hz 10 bursts with each burst of 100 Hz four pulses. EPSCs were recorded for 20 min after TBS application. Data was filtered at 1 KHz and analyzed with Clampfit software (Molecular devices, USA).

Immunohistochemical analysis: Mice were perfused with ice-cold phosphate buffered saline (PBS) and followed by 4% paraformaldehyde (PFA). Brains were dissected and analyzed by immunohistochemistry. For paraffin sections, brain hemispheres were embedded sagittally in paraffin and prepared into sections of 5 m slices. Sections were deparaffinized, and antigen retrieval was performed in citrate buffer (pH 6.0). For immunostaining, the sections were incubated with anti-Map2 (Millipore, Burlington, Mass.), anti-Tau (Cell signaling, Danvers, Mass.), anti-pNFh (Biolegend, San Diego, Calif.), anti-active caspase 3 (Cell signaling), anti-MBP (R&D systems, Minneapolis, Minn.), anti-LAMP1 (Abcam, Cambridge, UK), anti-LC3 (Cell signaling) or anti-Dcx (Abcam) antibodies. This step was followed by incubation with Alexa Fluor 488-conjugated anti-goat IgG (Thermo, Waltham, Mass.) or Alexa Fluor 555-conjugated anti-rabbit IgG secondary antibodies (Thermo). The sections were counterstained with DAPI. The immunofluorescent images were captured using a LSM700 Laser-Scanning confocal microscope (Carl Zeiss, Heildenheim, Germany). For diaminobenzidine (DAB) staining, immunohistochemistry was performed with the peroxidase substrate in the DAB kit (Vector Laboratories Inc., Burlingame, Calif.). NeuN-positive and active caspase 3-positive cells from cortex layer V were counted using Icy micromanager program (Institut Pasteur, Paris, France). The length of the Map2 and Tau positive dendrites and axons were measured using Icy program.

Cell culture: SH-SY5Y neuroblastoma cells were cultured at 37° C., 5% CO₂., in Dulbecco's Modified Eagle Medium/Ham's F-12 nutrient mixture (DMEM/F12) (Invitrogen, Carlsbad, Calif.) supplemented with 10% heat-inactivated fetal bovine serum containing 100 units/ml penicillin, 100 μg/ml streptomycin. Primary cortical neuron cultures were derived from embryo day 18 (E18) from ICR mice. The dissociated cells were plated on glass cover slips coated with poly-D-lysine in Neurobasal medium supplemented with 2% B27 (Invitrogen), 100 units/ml penicillin, 100 μg/ml streptomycin, and 2 mM L-glutamine. Primary NSCs from normal mice were isolated from the subventricular zone (SVZ) of 8 week-old C57BL/6 mice brain. NSCs from adult Tg2576 mice brain were obtained from Seoul National University Hospital. The neurospheres were cultured as previously described (M. Y. Kim, B. S. Moon, K. Y. Choi, Isolation and maintenance of cortical neural progenitor cells in vitro. Methods Mol Biol 1018, 3-10 (2013)). For neurosphere culture from normal mice, the cells were dissociated from brain tissue and grown in N2 medium in 25 cm² flasks (SPL, Gyeonggi-do, Korea) in suspension. bFGF (20 ng/ml, Peprotech, Princeton, N.J.) and human EGF (20 ng/ml, Peprotech) were added to the media to allow the cells to form neurospheres. For analyses, NSCs were cultured as a monolayer. For differentiation of NSCs, the neurospheres were dissociated with TrypLE (Invitrogen), plated on 15 μg/ml poly-L-ornithine-(Sigma-Aldrich, St. Louis, Mo.) and 10 μg/ml fibronectin (Gibco)-coated plates, and cultured in bFGF- and EGF-depleted N2 medium for 2 days. For neurosphere culture from Tg2576 mice, the cells were grown in DMEM/F12 supplemented with B27 supplements in 75 cm² flasks (SPL) in suspension. bFGF (10 ng/ml) and human EGF (10 ng/ml) were added to the media to allow the cells to form neurospheres. For inducing differentiation and AP expression, the neurospheres were dissociated with pipetting, plated on 15 μg/ml poly-L-ornithine- and 10 μg/ml fibronectin (Gibco)-coated plates, and cultured in bFGF- and EGF-depleted medium for 2 days.

Protein extraction and Western blotting: Cells and tissues were washed twice with ice-cold phosphate-buffered saline (PBS) and extracted by homogenizing with Ripa buffer (10 mM HEPES, 1.5 mM MgCl₂, 10 mM KCl, 0.01 M DTT, protease inhibitors, pH 7.9). Lysates were centrifuged at 13,000 rpm for 20 min at 4° C., and the protein content in the supernatant was determined using the Bradford assay (Bio-rad, Hercules, Calif.). For subcellular fractionation, cells were lysed with lysis buffer (250 mM Sucrose, 20 mM HEPES, pH 7.4, 10 mM KCl, 1.5 mM MgCl₂, 1 mM EGTA, 1 mM EDTA, 1 mM DTT) containing protease inhibitor for 30 min on ice, followed by centrifugation at 720 g for 5 min at 4° C. Supernatants were centrifuged at 15,000 rpm for 10 min at 4° C., and the resulting supernatant was used as the cytosol fraction. After centrifugation at 720 g for 5 min, pellets were washed with lysis buffer and dissolved in nuclear lysis buffer (50 mM Tris HCl, pH 8.0, 150 mM NaCl, 1% NP40, 0.5% sodium deoxycholate, 0.1% SDS, 10% glycerol) as the nuclear fraction. Protein from each sample was subjected to 8%˜15% SDS-PAGE, and the resolved proteins were transferred to nitrocellulose or polyvinylidene fluoride membrane. The membranes were blocked with 5% nonfat milk powder in Tris-buffered saline/Tween 20 (TBST) for 1 h at room temperature, then incubated with anti-phospho-ERK (Cell signaling), anti-ERK (Cell signaling), anti-LAMP1 (Abcam), anti-LC3 (Cell signaling), anti-cathepsin B (Cell signaling), anti-p62 (Cell signaling), anti-p62 (5114, Cell signaling), anti-phospho-mTOR (5536, Cell signaling), anti-mTOR (2983, Cell signaling), anti-phospho-ULK1 (14202, Cell signaling), anti-ULK1 (8054, Cell signaling), anti-TFEB (852501, Biolegend), and anti-GAPDH (Cell signaling) overnight at 4° C. After washing, membranes were incubated with horseradish peroxidase-conjugated goat anti-rabbit IgG antibody (Thermo) or goat anti-rat IgG antibody (Thermo) for 2 h at room temperature. Peroxidase activity was visualized with enhanced chemiluminescence. The detected signals were quantified using a LAS-4000 system (Fuji Film, Tokyo, Japan).

Quantitative PCR (qRT-PCR): Quantitative PCR analysis was performed on NSCs as previously described (J. Konirova et al., Modulated DISP3/PTCHD2 expression influences neural stem cell fate decisions. Sci Rep 7, 41597 (2017)). Total RNA was extracted from cells using TRIzol (Invitrogen). Reverse transcription was performed using M-MLV reverse transcriptase (Invitrogen). qRT-PCR was performed using the SYBR™ Green PCR master mix (Thermo) according to the manufacturer's guidelines. Results were expressed relative to the housekeeping gene GAPDH (Glyceraldehyde-3-Phosphate Dehydrogenase).

Immunocytochemistry: SH-SY5Y cells or adult NSCs were placed on glass coverslips coated with poly-L-ornithine/laminin or poly-D-lysine, respectively. After washing three times with PBS for 5 min, cells were fixed in 10% formalin for 15 min at room temperature. The cells were then washed with PBS and permeabilized in 0.1% Triton X-100 for 2 min. Cells were placed in blocking solution containing 5% BSA in PBS for 1 h at room temperature and incubated with anti-active caspase 3 (cell signaling), anti-Aβ (thermo), anti-Tau (Cell signaling), anti-LC3 (Cell signaling) and anti-LAMP1 (Abcam) in blocking buffer for 2 h at room temperature. After washing, cells were incubated with goat anti-rabbit antibody conjugated with Alexa Fluor 488 (Thermo) and/or goat anti-rat antibody conjugated with Alexa Fluor 555 (Thermo) overnight. After washing, cells were incubated with 4′,6-diamidino-2-phenylindole (DAPI) for 5 min. Coverslips were mounted using mounting medium (Biomeda, Foster City, Calif.) and visualized by confocal microscopy using a LSM700 microscope (Carl Zeiss). The percentage of coefficient was calculated using the pixels above threshold of fluorescence intensities. The intra-lysosomal pH was estimated using LysoTracker Red DND-99 (L7528, Invitrogen) following manufacturer's instructions. Cells were incubated with 500 nM for 30 min at 37° C. The fluorescence intensity was observed under a confocal microscopy using a LSM700 microscope (Carl Zeiss). The number of LysoTracker puncta was analyzed with Icy software.

Behavioral Test

Y-maze test: Animals were placed in the center of the Y-maze and their activity was recorded for 3 min. Y-maze is a three-arm maze with 1200 angles between all arms (40 cm long×15 cm high). Video tracking was performed using Smart video tracking software (Panlab, USA) and the order and number of entries into each arm were recorded. Spontaneous alternation was counted when a mouse made successive entries into the three arms in a row without visiting a previous arm.

Novel object recognition test: To test novel object recognition, mice were habituated in an empty open field arena (40 cm×40 cm). For the training trial, mice were placed in an open field arena with two identical objects for 10 min each. Next day, the test trial was performed for 3 min with one of the two familiar objects replaced with a new one. Video tracking was performed using Smart video tracking software (Panlab, USA) and recognition of familiar and novel objects was calculated as the percentage of time spent on new objects out of the time spent on exploring all objects.

Plasma cathepsin B level: Plasma was collected from 8-month and 13-month old 5XFAD mice using EDTA tubes and stored at −80° C. until use. Cathepsin B ELISA kit (Novus Biologicals, Centennial, Colo.) was used for analyzing cathepsin B levels in plasma. 100 μl of standard solution (from 0 to 10 ng/ml) and 100 μl of plasma were added to the 96-well plates and incubated for 90 min at 37° C. 100 μl of Biotinylated Detection Antibody working solution was immediately added to each well and incubated for 1 hour at 37° C. The solution from each well was decanted and washed with 350 μl of wash buffer 3 times. 100 μl of HRP Conjugate working solution was then added to each well and incubated for 30 min at 37° C. The solution was decanted from each well, and the washing process was repeated five times. 90 μl of Substrate Reagent was added to each well, and the plate was protected from light and incubated for about 15 min at 37° C. 50 μl of Stop Solution was added to each well, and the plate was read with a microplate reader set to 450 nm.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

1. A pharmaceutical composition comprising trametinib for the treatment of a patient diagnosed with neurodegenerative disease at a daily dose effective to induce change in the level of one or more markers in a biological sample obtained from the patient after at least four weeks' daily administration as compared to prior to administration.

2. The pharmaceutical composition of claim 1, wherein the daily dose is effective to induce change in the level of the one or more markers in the biological sample obtained from the patient of at least 1.3 fold, at least 1.5 fold, at least 2 fold, at least 3 fold, at least 4 fold, at least 5 fold, at least 6 fold, at least 7 fold, at least 8 fold, at least 9 fold, or at least 10 fold, at least 20 fold, at least 50 fold, or at least 100 fold.

3. The pharmaceutical composition of claim 1, wherein the daily dose is effective to decrease the level of the one or more markers in a biological sample obtained from the patient by at least 20%, by at least 30%, at least 40%, at least 50%, at least 60%, at least 70%, at least 80%, at least 90%, or at least 99% after the at least four weeks' administration compared to prior to administration.

4. The pharmaceutical composition of any one of claims 1-3, wherein each of the one or more markers is encoded by a human homolog of the mouse gene selected from the group consisting of: Gabrb1, Gabrr2, Glra3, Nr3c2, Cdk15, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Dcn, and Hmgb1.

5. The pharmaceutical composition of claim 4, wherein the human homolog is selected from the group consisting of GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, DISC1, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6V0C, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

6. The pharmaceutical composition of any one of claims 1-3, wherein each of the one or more markers is a protein related to lysosomal activity.

7. The pharmaceutical composition of claim 6, wherein the protein related to lysosomal activity is a cathepsin.

8. The pharmaceutical composition of claim 7, wherein the cathepsin is selected from the group consisting of: Cathepsin S, Cathepsin D, Cathepsin B, Cathepsin K, and Cathepsin L.

9. The pharmaceutical composition of any one of claims 1-8, wherein trametinib is administered at an oral dose between 0.5 and 2 mg/day.

10. The pharmaceutical composition of any one of claims 1-9, wherein the neurodegenerative disease is selected from the group consisting of Alzheimer's disease (AD), mild cognitive impairment (MCI), dementia, vascular dementia, senile dementia, frontotemporal dementia (FTD), Lewy body dementia (LBD), Parkinson's disease (PD), multiple system atrophy (MSA), corticobasal degeneration (CBD), progressive supranuclear palsy (PSP), Huntington's disease (HD), amyotrophic lateral sclerosis (ALS, Lou-Gehrig's disease), primary lateral sclerosis (PLS), progressive bulbar palsy (PBP), progressive muscular atrophy (PMA), pseudobulbar palsy, hereditary spastic paraplegia (HSP), cerebellar ataxia, Creutzfeldt-Jakob disease (CJD), multiple sclerosis (MS), and Guillain-Barre syndrome (GBS).

11. The pharmaceutical composition of claim 10, wherein the neurodegenerative disease is Alzheimer's disease (AD).

12. A pharmaceutical composition comprising trametinib for the treatment of a patient diagnosed with a disorder associated with lysosomal dysfunction or autophagic flux.

13. The pharmaceutical composition of claim 12, wherein the disorder is selected from the group consisting of: lysosome storage disease, spinocerebellar ataxia, oculopharyngeal muscular dystrophy, prion diseases, fatal familial insomnia, alpha-1 antitrypsin deficiency, dentatorubral pallidoluysian atrophy, x-linked spinobulbar muscular atrophy, neuronal intranuclear hyaline inclusion disease, multiple sclerosis, glaucoma and age-related macular degeneration.

14. The pharmaceutical composition of claim 13, wherein the lysosome storage disease is selected from the group consisting of: alpha-mannosidosis, aspartylglucosaminuria, juvenile Neuronal Ceroid Lipofuscinosis (JNCL, juvenile Batten or CLN3 Disease), cystinosis, Fabry Disease, Gaucher Disease Types I, II, and III, Glycogen Storage Disease II (Pompe Disease), GM2-Gangliosidosis Type I (Tay Sachs Disease), GM2-Gangliosidosis Type II (Sandhoff Disease), Metachromatic Leukodystrophy, Mucolipidosis Types I, II/III and IV, Mucopolysaccharide Storage Diseases (Hurler Disease and variants, Hunter, Sanfilippo Types A, B, C, D, Morquio Types A and B, Maroteaux-Lamy and Sly diseases), Niemann-Pick Disease Types A/B, C1 and C2, Schindler Disease Types I and II.

15. A pharmaceutical composition comprising trametinib for the treatment of a patient diagnosed with a disorder associated with neuronal injury.

16. The pharmaceutical composition of claim 15, wherein the disorder is selected from the group consisting of: glaucoma, stroke, head trauma, spinal injury, optic injury, ischemia, hypoxia, multiple sclerosis, and multiple system atrophy, diabetic neuropathies, virus-associated neuropathies, acquired immunodeficiency syndrome (AIDS) related neuropathy, infectious mononucleosis with polyneuritis, viral hepatitis with polyneuritis, Guillain-Barre syndrome, botulism-related neuropathy, toxic polyneuropathies including lead and alcohol-related neuropathies, nutritional neuropathies including subacute combined degeneration, angiopathic neuropathies including neuropathies associated with systemic lupus erythematosus, sarcoid-associated neuropathy, carcinomatous neuropathy, compression neuropathy, carpal tunnel syndrome, hereditary neuropathies, Charcot-Marie-Tooth disease, and peripheral nerve damage associated with spinal cord injury.

17. The pharmaceutical composition of claim 16, wherein the disorder is an ocular injury, ocular disorder, or optic neuropathy selected from the group consisting of: toxic amblyopia, optic atrophy, higher visual pathway lesions, disorders of ocular motility, third cranial nerve palsies, fourth cranial nerve palsies, sixth cranial nerve palsies, internuclear ophthalmoplegia, gaze palsies, eye damage from free radicals, ischemic optic neuropathies, toxic optic neuropathies, ocular ischemic syndrome, optic nerve inflammation, infection of the optic nerve, optic neuritis, optic neuropathy, papilledema, papillitis, retrobulbar neuritis, commotio retinae, glaucoma, macular degeneration, retinitis pigmentosa, retinal detachment, retinal tears or holes, diabetic retinopathy, iatrogenic retinopathy, and optic nerve drusen.

18. A pharmaceutical composition comprising trametinib for the treatment of a patient diagnosed with a disorder associated with damaged myelin or demyelination of nerve fibers.

19. The pharmaceutical composition of claim 18, wherein the disorder is selected from the group consisting of: multiple sclerosis, acute disseminated encephalomyelitis, transverse myelitis, Schilder's disease, Balo's disease, clinically isolated syndrome, Alexander's disease, Canavan disease, Cockayne's syndrome, Pelizaeus-Merzbacher disease, optic neuritis, neuromyelitis optica, HTLV-I associated myelopathy, hereditary leukoencephalopathy, Guillain-Barre syndrome, central pontine myelinolysis, deep white matter ischemia, progressive multifocal leukoencephalopathy, demyelinating HIV encephalitis, demyelinating radiation injury, acquired toxic-metabolic disorders, posterior reversible encephalopathy syndrome, central pontine myelinolysis, leukodystrophies, adrenoleukodystrophy, Krabbe's globoid cell and/or metachromatic leukodystrophy, cervical spondylotic myelopathy resulting from cervical stenosis, traumatic injury to the brain or spinal cord, stroke and neonatal hypoxic injury.

20. A composition for use in determining therapeutic efficacy of a MEK 1/2 inhibitor on a neurodegenerative disease, a disorder associated with lysosomal dysfunction or autophagic flux, a disorder associated with neuronal injury, or a disorder associated with damaged myelin or demyelination of nerve fibers, comprising a probe or an antibody that specifically binds to a marker encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fe-1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Den, and Hmgb1.

21. The composition of claim 20, wherein the human homolog is selected from the group consisting of GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, DISC1, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6V0C, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

22. A composition for use in determining therapeutic efficacy of a MEK 1/2 inhibitor on a neurodegenerative disease, a disorder associated with lysosomal dysfunction or autophagic flux, a disorder associated with neuronal injury, or a disorder associated with damaged myelin or demyelination of nerve fibers, comprising an antibody that specifically binds to a marker protein related to lysosomal activity.

23. The composition of claim 22, wherein the marker protein related to lysosomal activity is a cathepsin.

24. The composition of claim 23, wherein the cathepsin is selected from the group consisting of: Cathepsin S, Cathepsin D, Cathepsin B, Cathepsin K, and Cathepsin L.

25. The composition of any one of claims 20-24, wherein the MEK 1/2 inhibitor is trametinib.

26. The composition of any one of claims 20-25, wherein the therapeutic efficacy of the MEK 1/2 inhibitor is determined by comparing the level of the one or more markers in a biological sample obtained from a patient diagnosed with said disease or disorder after administration of trametinib (a) to the level of the one or more markers in a biological sample obtained from the patient prior to commencing administration of trametinib or (b) to the level of the one or more markers in a biological sample obtained from healthy subjects who are free of the disease or disorder.

27. A method of detecting the level of a marker using a probe or an antibody that specifically binds to the marker in a biological sample obtained from a patient diagnosed with a disorder selected from a neurodegenerative disease, a disorder associated with lysosomal dysfunction or autophagic flux, a disorder associated with neuronal injury, or a disorder associated with damaged myelin or demyelination of nerve fibers, to provide information on therapeutic efficacy of an MEK 1/2 inhibitor on the disorder, wherein the marker is encoded by a human homolog of the mouse gene selected from the group consisting of: Gabrb1, Gabrr2, Glra3, Nr3c2, Cdk15, Grin2a, Grin2b, Plcxd3, Chrm2, Chrna3, Chrna7, Chrnb2, Nefl, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Il1rapl1, Cpeb3, Tnfrsf12a, Hspb1, Oprm1, Lmx1a, Clcf1, Aspm, Mecp2, Ntf3, Vegfa, Lrp2, Fez1, Atp6v0c, Rnase6, Ctsk, Acr, Prss16, Lamp5, Prdx6, Unc13d, Bag3, Tial1, Adrb2, Hps4, Ass1, Cckar, Gimap5, Hmox1, Sesn3, Pcsk9, Capn1, Rnf152, Vps13c, Den, and Hmgb1.

28. The method of claim 27, wherein the human homolog is selected from the group consisting of GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, DISC1, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ), ATP6V0C, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

29. A method of detecting the level of a marker using a probe or an antibody that specifically binds to the marker in a biological sample obtained from a patient diagnosed with a disorder selected from a neurodegenerative disease, a disorder associated with lysosomal dysfunction or autophagic flux, a disorder associated with neuronal injury, or a disorder associated with damaged myelin or demyelination of nerve fibers, to provide information on therapeutic efficacy of a MEK 1/2 inhibitor on the disorder, wherein the marker is a protein related to lysosomal activity.

30. The method of claim 29, wherein the protein related to lysosomal activity is a cathepsin.

31. The method of claim 30, wherein the cathepsin is selected from the group consisting of: Cathepsin S, Cathepsin D, Cathepsin B, Cathepsin K, and Cathepsin L.

32. The method of any one of claims 27-31 wherein the MEK 1/2 inhibitor is trametinib.
